(19) United States
(12) Patent Application Publication
(10) Pub. No.: US 2011/0262502 A1
(43) Pub. Date: Oct. 27, 2011

(54) PULMONARY DELIVERY OF 17-HYDROXYPROGESTERONE CAPROATE (17-HPC)

(75) Inventors: Chang LEE, Bethesda, MD (US); Tao Tom Du, North Potomac, MD (US)

(73) Assignee: Prairie Pharmaceuticals LLC, Bethesda, MD (US)

(21) Appl. No.: 13/174,939

(22) Filed: Jul. 1, 2011

Related U.S. Application Data
(63) Continuation-in-part of application No. 13/021,950, filed on Feb. 7, 2011.
(60) Provisional application No. 61/302,325, filed on Feb. 8, 2010.

(51) Int. Cl. A61K 31/57; A61K 9/72; A61K 9/12; A61P 11/00; A61P 31/04; A61P 33/00; A61P 37/08; A61P 19/02; A61K 9/14; A61P 25/00; B82Y 5/00

(52) U.S. Cl. 424/400; 514/179; 424/46; 514/171; 977/775

(57) ABSTRACT

The invention relates to 17-HPC pulmonary formulations for administration by inhalation comprising 17-HPC and a pharmaceutically acceptable excipient. Particle size reduction of 17-HPC is required for the pulmonary delivery, and can be achieved with a surfactant or water without the surfactant. Preferred pulmonary formulations include a powder blend comprising a therapeutically effective amount of at least one steroid hormone (progestogen) as a glucocorticoid sensitizer, and at least one pharmaceutically acceptable excipient, wherein the at least one steroid hormone (progestogen) has a particle size distribution profile ranging from about one nanometer to about ten microns in the powder blend.

### CROSS-REFERENCE TO RELATED APPLICATIONS

This application is a Continuation-In-Part application of U.S. patent application Ser. No. 13/021,950 filed Feb. 7, 2011, now pending, and claims the benefit of PCT International Patent Application No. PCT/US11/23917, filed Feb. 7, 2011, and U.S. Provisional Patent Application No. 61/302,325, filed on Feb. 8, 2010, the entire disclosures of which are incorporated herein by reference.

### FIELD OF THE INVENTION

Glucocorticoid insensitivity presents a profound management problem in diseases/conditions treated with glucocorticoids because the therapy is not effective. The present invention relates, inter alia, to inhalation formulations comprising a progesterone such as 17alpha-hydroxyprogesterone caproate (17-HPC); and methods and kits for administering a progestogen as a glucocorticoid sensitizer to restore corticosteroid sensitivity or reverse the glucocorticoid insensitivity or enhance glucocorticoid sensitivity, in order to treat one or more glucocorticoid insensitivity related diseases or conditions. For example, the present invention relates to inhalation formulations and methods for reversing the glucocorticoid insensitivity in a subject having no history of menstrual cycle-related exacerbation or allergy to self-hormones, particularly progesterone, such as premenstrual or perimenstrual deterioration in the symptoms, e.g., premenstrual worsening of atopic dermatitis or premenstrual exacerbations of asthma, and exhibiting relatively or totally refractory responses to glucocorticoid therapy, e.g., glucocorticoid resistance. The formulations, methods and kits of the present invention provide for the administration of a sex hormone to the subject who is corticosteroid dependent or corticoid resistant or unresponsive or intolerant to corticosteroids.

### BACKGROUND OF THE INVENTION

Glucocorticoids are the first-line treatment for various immune-inflammatory and allergic diseases. For example, the autoimmune diseases include more than 70 chronic disorders that affect about 5% of the US population, and include those that most occur in women (>80%) such as Sjogren's syndrome, SLE, autoimmune thyroid disease (Hashimoto's thyroiditis and well as Graves' disease) and scleroderma, or relatively common among women (60-75%) such as rheumatoid arthritis (RA), multiple sclerosis (MS) and myasthenia gravis; or those that occur at a similar female:male ratio such as sarcoid and inflammatory bowel diseases. Glucocorticoid insensitivity presents a profound management problem in those diseases/conditions treated with steroids, and twenty to forty percent of patients may fail to achieve disease control. The glucocorticoid insensitivity may present as relatively or totally refractory to glucocorticoid therapy; unresponsive or intolerant to corticosteroids; unresponsive to an adequate induction dose of corticosteroids; initially responsive to corticosteroids but relapses quickly upon drug withdrawal or dose tapering (corticosteroid dependent); corticoid resistant, e.g., requires a very high dose treatment; or “difficult to treat” or severe condition. For example, 20-30% of patients with severe and steroid-resistant Crohn's Disease will not respond to steroid therapy (Michetti P, Mottet C, Juillerat P, Felley C, Vader J-P, Burnand B, Gonvers J-J, Froehlich F: Severe and Steroid-Resistant Crohn's Disease. Digestion 2005; 71:19-25).

Diseases/conditions related to glucocorticoid insensitivity may include: refractory inflammatory bowel disease, such as Refractory ulcerative colitis and children with severe Crohn disease, corticosteroid refractory asthma or glucocorticoid resistant asthma or symptomatic corticosteroid dependent asthma, desquamative interstitial pneumonia refractory to corticosteroid, refractory inflammatory myopathies, refractory myasthenia gravis, refractory pemphigus vulgaris, methotrexaterefractory RA patients, refractory nephrotic syndrome in adults, corticosteroid dependent systemic lupus erythematosus (SLE), primary Sjogren's syndrome, systemic vasculitis and polymyositis, chronic graft-versus-host disease, corticosteroid dependent or refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, critically ill patients with sepsis or acute respiratory distress syndrome (ARDS) or relative adrenal insufficiency, corticosteroid-dependent conditions (e.g., rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome etc.). Glucocorticoid insensitivity has serious health, societal, and economic costs. For example, a small percentage of patients with asthma (5-10%) have severe corticosteroid-refractory condition that often fails to respond but these patients account for >50% of the total asthma health care costs.

Glucocorticoids suppress inflammation mainly as a result of both activation of anti-inflammatory genes and suppression of pro-inflammatory genes. The activation of anti-inflammatory gene expression starts as glucocorticoid binds cytosolic glucocorticoid receptor (GR), which is activated and translocates to the nucleus. Once in the nucleus, it binds to glucocorticoid response elements (GREs) and transcriptional coactivator molecules, and causes acetylation of core histones, which leads to the expression of anti-inflammatory genes. Inflammatory stimuli switch on multiple inflammatory genes that encode cytokines, chemokines, adhesion molecules, inflammatory enzymes, and receptors via pro-inflammatory transcription factors, such as nuclear factor κB (NFκB) and activator protein 1, and the recruitment of co-repressor molecules. Activated glucocorticoid receptors bind to the coactivators in the nucleus to inhibit histone acetyltransferase (HAT) activity directly and recruit histone deacetylase 2 (HDAC2), leading to suppression of the activated inflammatory genes.

Several possible molecular mechanisms of glucocorticoid resistance have been recognized, and include genetic susceptibility, lack of or defective binding to GR and translocation, reduced GR expression, lack of co-repressor activity, or enhanced activation of inflammatory pathways. For example, glucocorticoid receptors might be phosphorylated by several kinases (e.g., p38 mitogen-activated protein kinase, c-Jun N-terminal kinase, and extracellular signal-regulated kinase) that results in the defective binding, alterations in their stability, translocation to the nucleus, binding to DNA, and interaction with other proteins. Excessive activation of the transcription factor activator protein 1 can prevent GRs binding to glucocorticoid response elements (GREs) or inhibiting nuclear factor κB; Nitric oxide (NO) can nitrate tyrosine residues on GRs; GRs can also be ubiquitinated (Ub), which results in degradation of GR by the proteasome; reduced histone deacetylase-2 (HDAC2) expression, raised macrophage migration inhibitory factor, and increased P-glycoprotein-mediated drug efflux (Peter J Barnes, Ian M Adcock. Glucocorticoid resistance in inflammatory diseases. Lancet 2009; 373: 1905-17).

The clinical and biological mechanisms of steroid-dependency are not well understood compared with those determining steroid-resistance. Steroid-dependency and steroid-resistance may share some common intrinsic mechanisms while other mechanisms are simply clinical or pharmacological.

Many attempts have been made to ameliorate the effects of glucocorticoid insensitivity. A common approach is to use broad-spectrum anti-inflammatory treatments such as immunosuppressive or immunomodulators agents (e.g., cyclosporine, methotrexate, gold, 6-mercaptopurine, biologic products such as intravenous immunoglobulin and Mepolizumab), and calcineurin inhibitors (e.g., cyclosporin, tacrolimus). Various approaches have been proposed or developed to reverse glucocorticoid resistance such as p38 MAP kinase inhibitors, JNK inhibitors (decrease AP1), Vitamin D in steroid-resistant asthma (increase regulatory T cells), MIF inhibitors, Histone deacetylate-2 activators, Theophylline, Phosphoinositide-3-kinase-ε inhibitors, antioxidants, iNOS inhibitors and P-glycoprotein inhibitors. The use of progestogen for reversing the glucocorticoid-insensitivity has not been discussed or presented anywhere, and the present invention represents a significant, surprising and unexpected advance in the art.

The different approaches for management of glucocorticoid insensitivity have had limited success. Some agents may work in a condition, but not others. Methotrexate is effective for rheumatoid arthritis, but it might be ineffective in cases of glucocorticoid-resistant inflammatory bowel disease caused by increased P-glycoprotein expression. Similarly, calcineurin inhibitors are useful in some patients with glucocorticoid-resistant inflammatory bowel disease, but they have not proven to be effective in glucocorticoid-resistant asthma. Further, the uses of those agents are often associated with significant adverse events. A high percentage of patients (60-70%) may fail treatment with methotrexate because of side effects. Phosphodiesterase-4 inhibitors for COPD and inflammatory conditions have dose-limiting side-effects of nausea, diarrhea, and headaches. Significant toxicity and side-effects have hampered the drug development programs for p38 MAP-kinase inhibitors and selective inhibitors to block inhibition of NFκB kinase (IKKβ)/NFκB (Peter J Barnes, Ian M Adcock. Glucocorticoid resistance in inflammatory diseases. Lancet 2009; 373: 1905-17).

Given that a considerable proportion of patients with autoimmune, allergic, and lymphoproliferative diseases are refractory to glucocorticoid therapy as well many different inflammatory diseases share similar molecular mechanisms in glucocorticoid insensitivity, there exists a heretofore unmet need in the art for methods for, developing a common therapeutic strategy to reverse the steroid-insensitivity. The use of progestogen, in accordance with the present invention, has been discovered to present a surprising, unexpected, and also practicable method to help patients with diseases/conditions that are unresponsive or intolerant to corticosteroids or corticosteroid dependent and resistant.

Progestogen products have been extensively used in a wide range of reproductive diseases/conditions for more than 60 years, and known to have anti-inflammatory effects. The majority of studies related to inflammatory responses were conducted in pregnancy-associated models. Progesterone/PR Maintains Uterine Quiescence via Antiinflammatory Actions (Carole R. Mendelson. Minireview: Fetal-Maternal Hormonal Signaling in Pregnancy and Labor Molecular Endocrinology 23: 947-954, 2009). Gellersen (2009) provided a comprehensive review of non-genomic progesterone actions, and summarized possible mechanisms of progesterone anti-inflammatory effects, including that progesterone opposes prostaglandin production in the uterus of pregnancy, partially by inhibiting cyclooxygenase (COX-2) expression; immunoregulatory function in human T-lymphocytes via G-protein activation and K+ channel Inhibition; progesterone-induced blocking factor (PIBF) acts on the phospholipase A2 enzyme, interferes with arachidonic acid metabolism, induces a Th2 biased immune response, and exerts an anti-abortive effect by controlling NK activity (Gellersen B et al. Non-genomic progesterone actions in female reproduction Human Reproduction Update, Vol. 15, No. 1 pp. 119-138, 2009). Another review by Challies (2009) suggests other possible mechanisms: progesterone blocks mitogen-stimulated lymphocyte proliferation, modulates antibody production, decreases the oxidative burst of monocytes, reduces the production of proinflammatory cytokines by macrophages in response to bacterial products, and alters cytokine secretion of T-cell clones to favor IL-10 production, upregulates Toll-like receptor 4 (TLR-4) expression and suppresses TLR-2 response to infection in intrauterine tissues, resulting in a protective role with respect to preterm delivery, inhibits basal and cytokine-enhanced matrix metalloproteinases (MMP)-1 and MMP-3 expression in cultured decidual cells demonstrating protection against preterm delivery (Challis J R et al. Inflammation and Pregnancy Reproductive Sciences 2009; 16; 206). Since the concept of using progestogen for reversing the glucocorticoid-insensitivity has not been disclosed, taught, suggested, discussed, or presented anywhere, the present discovery represents a significant and unexpected advance in the art.

Menstrual cycle-related exacerbation of common medical conditions is a well-recognized phenomenon, and may include migraine, epilepsy, asthma, irritable bowel syndrome, autoimmune progesterone dermatitis and stomatitis, and diabetes. Exacerbation is influenced by hormonal changes of the menstrual cycle. The majority of these effects occur during the luteal and menstrual phases of the cycle. For example, premenstrual asthma denotes worsening of asthma symptoms shortly before and/or during menstruation. Accurate documentation of symptoms on a menstrual calendar allows identification of women with cyclic alterations in disease activity. Female sex-steroid hormones play an important role but the exact mechanism is still unknown. Several theories exist to explain these menstrual cycle-related effects. These include fluctuations in levels of sex steroids, cyclic alterations in the immune system, increased airway hyperresponsiveness, changing perceptions of disease severity brought about by premenstrual alterations in mood, as seen in premenstrual syndrome, and allergy to self-hormones particularly progesterone. Menstrual cycle-related exacerbation might be ameliorated by progesterone supplementation [Allison M. Case and Robert L. Reid. Menstrual cycle effects on common medical conditions. Journal Comprehensive Therapy Issue Volume 27, Number 1/March, 2001; Beynon H L. Severe premenstrual exacerbations of asthma: effect of intramuscular progesterone. Lancet-13-AUG-1988; 2(8607): 370-2; Roby, Russell R et al. Sublingual progesterone dilutions as bronchodilator in asthmatic females. World Allergy Organization Journal: November 2007-Volume-Issue-p S148].

Glucocorticoid insensitivity often correlates with other factors believed to contribute to relatively or totally refractory responses to glucocorticoid therapy. These include the various risk factors noted above such as genetic susceptibility, abnormalities in the glucocorticoid receptor gene, viral infection and oxidative stress. For example, oxidative DNA damage is known to be a primary cause of the process of mutation and a leading cause of aging, cancer and other diseases because guanine, one of the four basic nucleotides that make up DNA and form the genetic code of life, is particularly sensitive to oxidative damage, and a predominant number of genetic mutations are linked to guanine. Thus, there exists a need in the art for methods for reducing the occurrence of glucocorticoid insensitivity related conditions (e.g., refractory asthma, refractory rheumatoid arthritis, refractory inflammatory bowel disease, chronic obstructive pulmonary disease and acute respiratory distress syndrome) associated with such risk factors.

A menstrual rhythm has been documented for exacerbations of asthma, which may have important clinical relevance to the patient with severe asthma. Beynon et al. (1988) reported 3 cases of severe premenstrual exacerbations of asthma that were treated with intramuscular progesterone. The patients hadn't responded to conventional treatment, including high-dose corticosteroids. In all cases there was a fall premenstrually in peak flow rate. The addition of intramuscular progesterone (100 mg daily in two cases and 600 mg twice a week in one) to the regimen eliminated the premenstrual dips in peak flow, and daily doses of prednisolone were reduced in the three patients. The above-described study and results are described in Beynon et al. (Severe premenstrual exacerbations of asthma: effect of intramuscular progesterone. Lancet-13-AUG-1988; 2(8607): 370-2.).

In another study, Russell R. et al. (2007) tested the hypothesis that pre-menstrual asthma is associated with allergy to self-hormones particularly progesterone by using sublingual progesterone dilutions as bronchodilator. Sixteen females who had a previous diagnosis of severe asthma and who were nebulization dependent were selected for the study. Spirometric studies were performed on these subjects. Study showed changes over time of the forced expiratory volume in one second (FEV1), the forced vital capacity (FVC), and the peak expiratory flow (PEF) measured at three times: (1) before treatment, (2) after sublingual normal saline treatment (3) after sublingual progesterone treatment. After treatment with sublingual progesterone, twelve of the sixteen patients (75%) experienced a bronchodilator effect (greater than 12% increase) in either FEV1 or FVC. Eight (50%) experienced an increase in both FEV1 and FVC. Eight (50%) had an increase of 27% or greater in PEF. The above-described study and results are described in Russell R et al. (Sublingual progesterone dilutions as bronchodilator in asthmatic females. World Allergy Organization Journal: November 2007-Volume-Issue-p S148.).

Activation of mitogen-activated protein kinases (MAPKs) is a critical event in mitogenic signal transduction. Ruzycky A L (1996) determined the effects of 17 beta-estradiol and progesterone on mitogen-activated protein kinase expression and activity. MAPK expression and activity was examined in uterine smooth muscle from rats pretreated with estradiol-17 beta alone or with estradiol-17 beta and progesterone. MAPK expression was detected by immunoblotting using erk1/2 antibodies. MAPK activity was detected by measurement of the phosphorylation of a MAPK-specific peptide sequence of myelin basic protein. Steroid treatment caused a modest (20%) decline in erk 1 and 2 expression in membrane and cytosolic fractions. Both estrogen and progesterone increased MAPK tyrosine phosphorylation and membrane-associated MAPK activity. Steroid treatment increased cytosolic MAPK tyrosine phosphorylation, but not enzymatic activity. The above-described study and results are described in Ruzycky A L (Effects of 17 beta-estradiol and progesterone on mitogen-activated protein kinase expression and activity in rat uterine smooth muscle. Eur. J. Pharmacol. 1996 Apr. 11; 300(3):247-54).

### SUMMARY OF THE INVENTION

Certain embodiments of the present invention are directed to inhalation formulations comprising a progesterone such as 17alpha-hydroxyprogesterone caproate (17-HPC) for pulmonary delivery.

Other embodiments of the present invention are directed to methods for restoring corticosteroid sensitivity or reversing the glucocorticoid insensitivity or enhancing glucocorticoid sensitivity.

Still other embodiments of the present invention are directed to methods for administering a pharmaceutical composition comprising a steroid hormone to a subject having no history of menstrual cycle-related exacerbation, and suffering from one or more glucocorticoid insensitivity related conditions. Glucocorticoid insensitivity related conditions include, for instance, a range of corticoid resistant diseases and immune-inflammatory disorders treated with steroids when the therapy becomes ineffective or intolerant or dependent or unresponsive or refractory to corticosteroids, and combinations thereof.

In one embodiment, a method of the present invention comprises administering a pharmaceutical composition comprising a steroid hormone to a subject having no history of menstrual cycle-related exacerbation, wherein the subject is at risk for developing glucocorticoid insensitivity due to exposure to oxidative stress.

Yet other embodiments of the present invention are directed to methods for restoring corticosteroid sensitivity or reversing the glucocorticoid insensitivity or enhancing glucocorticoid sensitivity, and treating one or more conditions selected from the group consisting of corticoid resistant diseases, corticosteroid refractory, corticosteroid-dependent immune-inflammatory disorders, and combinations thereof. Certain exemplary glucocorticoid resistant conditions include, but are not limited to, glucocorticoid resistant asthma, refractory rheumatoid arthritis, refractory inflammatory bowel disease, chronic obstructive pulmonary disease and acute respiratory distress syndrome, interstitial pulmonary fibrosis, and cystic fibrosis. Certain exemplary glucocorticoid refractory conditions include, but are not limited to, refractory ulcerative colitis, children with severe Crohn disease, corticosteroid refractory asthma, desquamative interstitial pneumonia refractory to corticosteroid, refractory inflammatory myopathies, refractory myasthenia gravis, refractory pemphigus vulgaris, methotrexate-refractory RA patients, refractory nephrotic syndrome, refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, critically ill patients with sepsis or acute respiratory distress syndrome (ARDS) and relative adrenal insufficiency. Certain exemplary glucocorticoid dependent conditions include, but are not limited to, rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, corticosteroid dependent systemic lupus erythematosus, corticosteroid dependent multiple sclerosis, symptomatic corticosteroid dependent asthma, primary Sjogren's syndrome, systemic vasculitis and polymyositis, organ transplants, graft-versus-host disease, and glucocorticoid dependent cancer.

Still other embodiments of the present invention are directed to kits comprising (i) a pharmaceutical composition comprising a steroid hormone and one or more pharmaceutically acceptable excipients; and (ii) instructions for administering the pharmaceutical composition to a subject preferably having no history of menstrual cycle-related exacerbation, and suffering from one or more glucocorticoid insensitivity related conditions. Glucocorticoid insensitivity related conditions include, for instance, a range of corticoid resistant diseases and immune-inflammatory disorders treated with steroids when the therapy becomes ineffective or intolerant or dependent or unresponsive or refractory to corticosteroids, and combinations thereof.

In another embodiment, the kits of the present invention comprise (i) a pharmaceutical composition comprising a steroid hormone and one or more pharmaceutically acceptable excipients; and (ii) instructions for administering the pharmaceutical composition to a subject who is at high risk to develop glucocorticoid insensitivity, but preferably has no history of menstrual cycle-related exacerbation, and preferably is at risk for developing one or more glucocorticoid insensitivity related conditions due to oxidative stress.

Yet other embodiments of the present invention are directed to kits comprising (i) a pharmaceutical composition comprising a steroid hormone and one or more pharmaceutically acceptable excipients; and (ii) instructions for administering the pharmaceutical composition to a subject that preferably has no history of menstrual cycle-related exacerbation, and wherein the subject is suffering from one or more glucocorticoid insensitivity related conditions in order to achieve the glucocorticoid-sensitizer effects of steroid-sparing in corticosteroid-dependent patients, better responsiveness or tolerance to corticosteroids, achieving efficacy by using a lower dose of corticosteroid, preventing individuals at risk for developing corticosteroid refractory responses or resistance or exacerbations in response to antigen exposures, infections, exercise, or irritants, achieving optimal immune-functions, easier responses for the subject or patient when steroid administration is tapered or withdrawn, or in prolonged administration of corticosteroids, decreased risks for developing corticosteroid-related adverse events such as opportunistic infections and bone loss, and combinations thereof.

It is to be understood that the embodiments described above are provided as representative embodiments of the present invention, and in no way are to be construed as limiting the scope of the present invention.

### BRIEF DESCRIPTION OF THE DRAWINGS

FIGS. 1-22 are graphical depictions of the results of several examples which illustrate certain embodiments of the invention, but in no way limit the scope of the invention.

FIG. 1 depicts IL-2 levels at baseline, after PHA stimulation and dose-dependent inhibition of IL-2 by dexamethasone.

FIG. 2 depicts that addition of IL-2 and IL-4 induces steroid resistance.

FIG. 3 depicts progestogen's effects (% Imax) in reversing steroid resistance: comparing 17HPC, P4 and MPA under low dose Dexamethasone (hereinafter, “Dexamethasone”) (10⁻¹⁰ M).

FIG. 4 depicts progestogen's effects (% Imax) in reversing steroid resistance: comparing 17HPC, P4 and MPA under high dose Dexamethasone (10⁻⁶ M).

FIG. 5 depicts that 17HPC restores corticosteroid sensitivity.

FIG. 6 depicts that 17HPC reverses steroid resistance and individual response patterns.

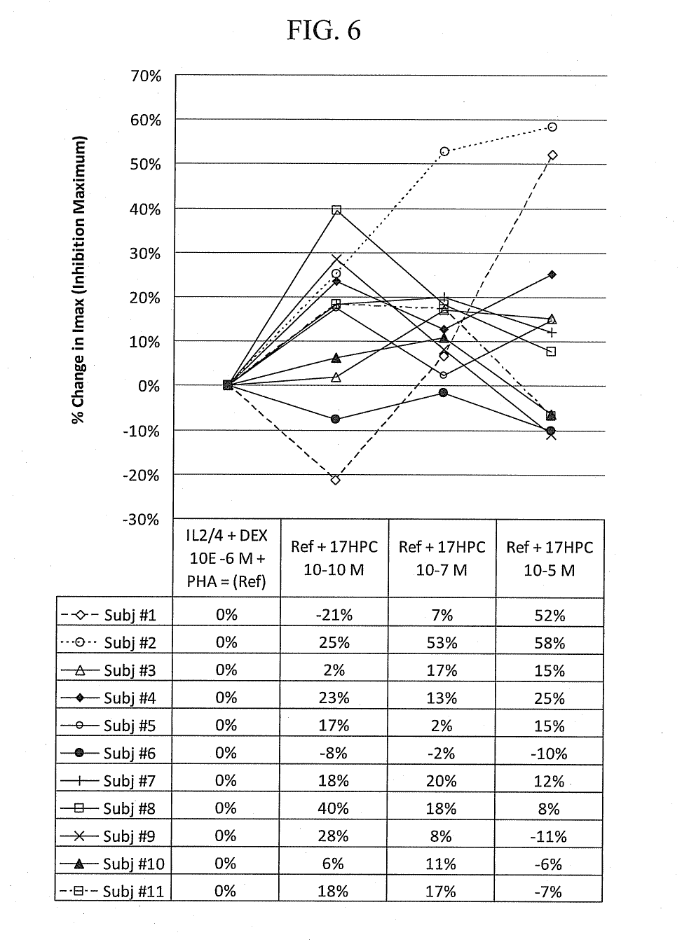

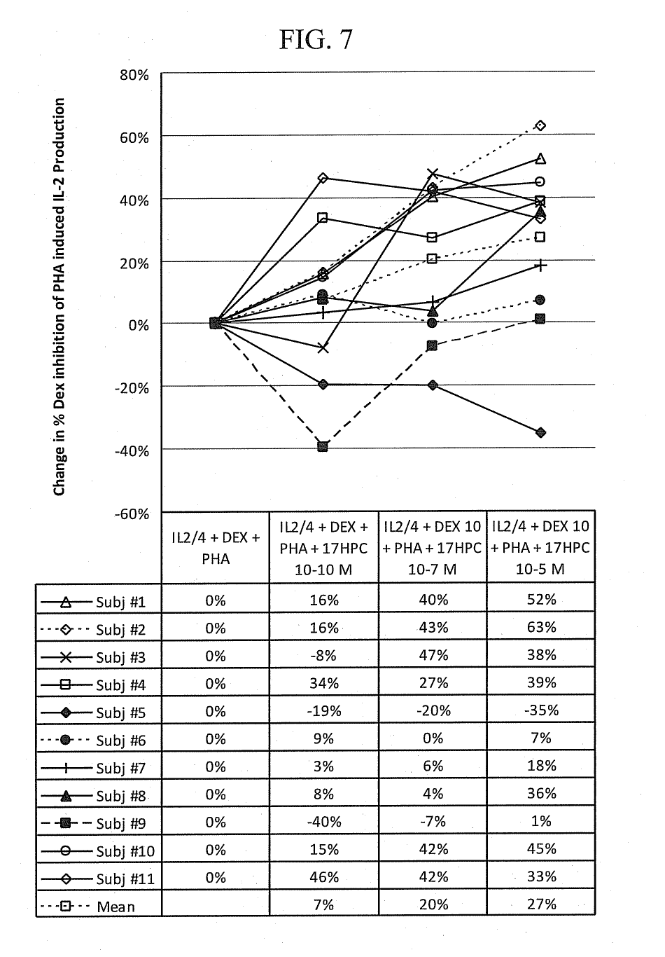

The results depicted in FIG. 7 show that 9 out 11 subjects had a more than 10% improvement in maximal Dexamethasone inhibition after receiving a dose of 17HPC, which is consistent with the results presented in FIG. 6.

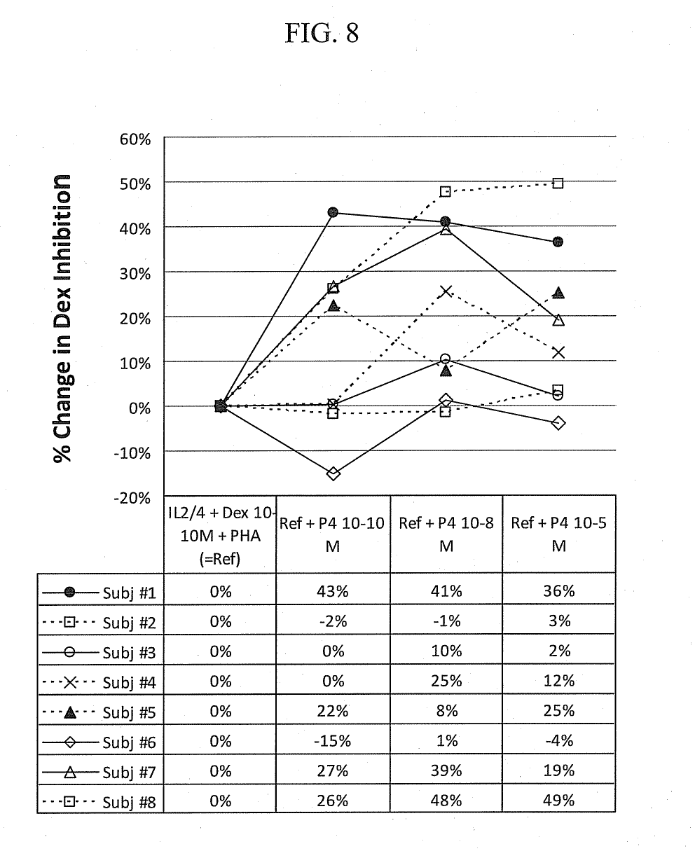

The results depicted in FIG. 8 show that 6 out of 8 subjects had a more than 10% improvement in maximal Dexamethasone inhibition after receiving a dose of natural progesterone, which is similar to 17HPC.

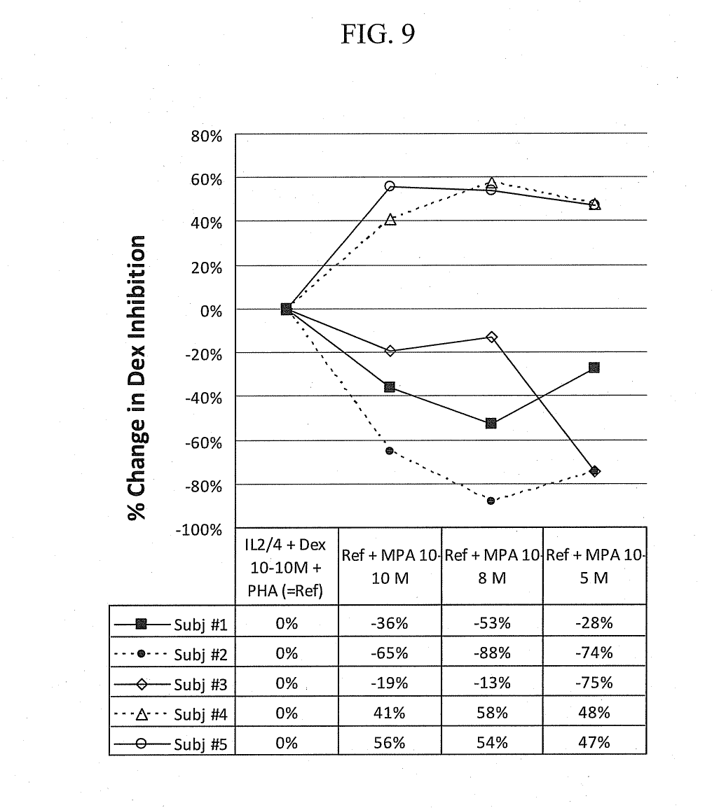

The results depicted in FIG. 9 shows that MPA treatment leads a total different response pattern: a “split” response. A sub-group had a great improvement up to 58% while another sub-group presented with a worsening in corticosteroid sensitivity, a reduction up to 88%.

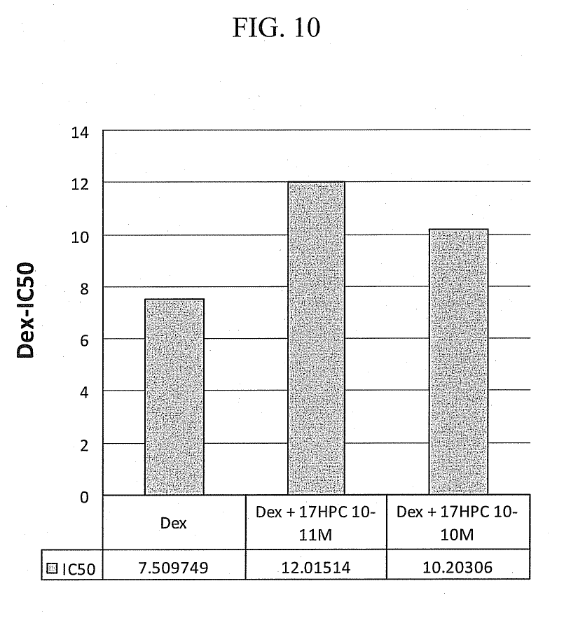

The effects of 17HPC on dexamethasone sensitivity measured by IL-2 inhibition in smokers are shown in Table 1 and FIG. 10: FIG. 10 shows add-on effect of 17HPC is improvement of steroid sensitivity.

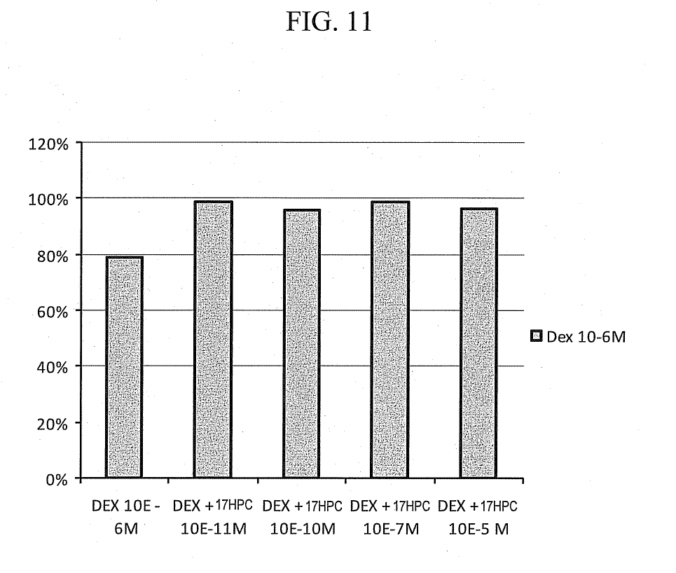

The maximal anti-inflammatory effect of Dexamethasone is 78% inhibition of PHA-induced IL-2 production at 10⁻⁶ M. The ‘add-on’ treatment of 17HPC produces a significantly better responsiveness and results in near 100% suppression of PHA induced IL-2 (FIG. 11). FIG. 11 thus depicts a better treatment responsiveness with the 17HPC add-on.

The combination of 17HPC with Dexamethasone consistently increases dexamethasone's anti-inflammatory effects, and is better than their uses individually.

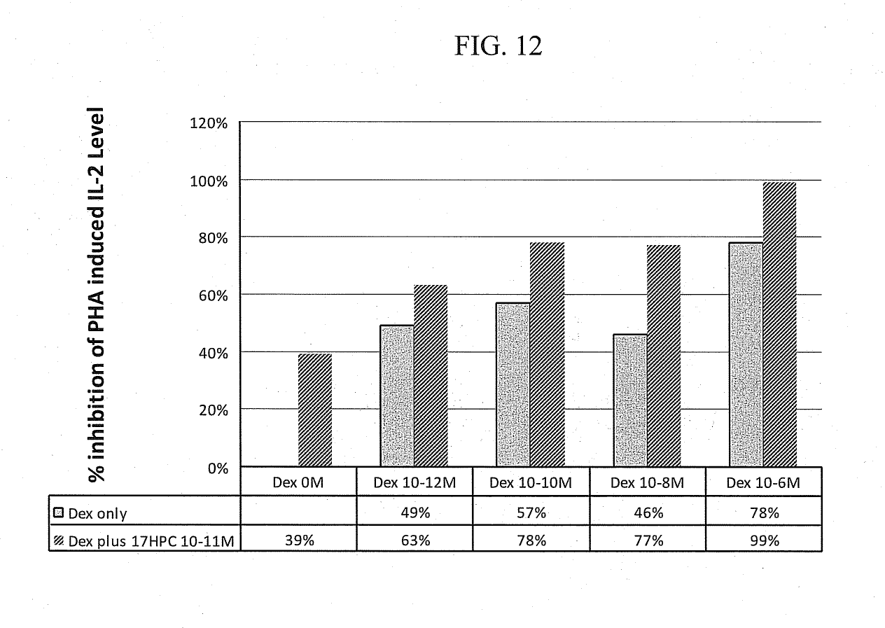

FIG. 12 shows that the combination leads to a synergetic effect, with 25-37% improvements in Dexamethasone efficacy. FIG. 12 thus depicts synergetic effects of combination of 17HPC with Dexamethasone.

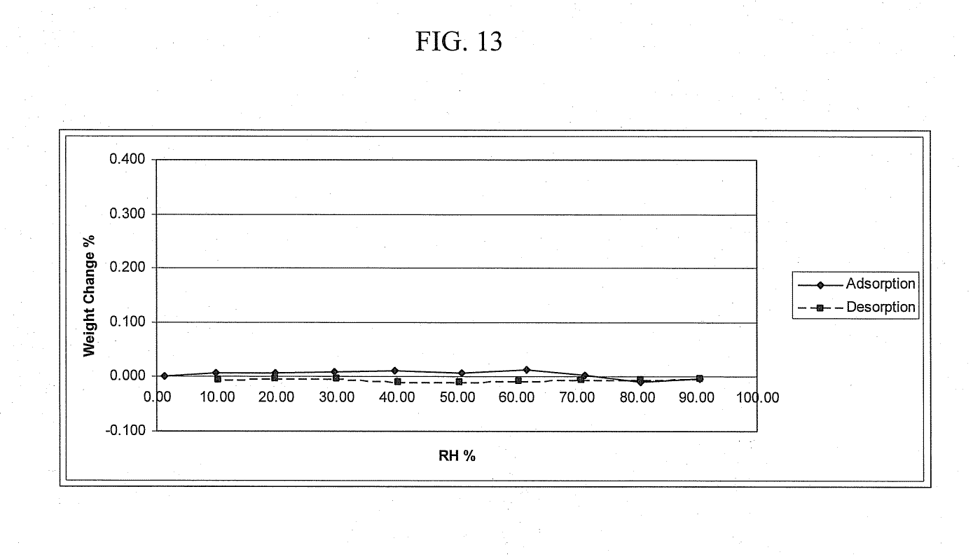

FIG. 13 depicts exemplary results of characterization of the bulk material 17-HPC (showing percent change in weight of the bulk material relative to percent change in relative humidity, or RH %) in a Dynamic Vapor Sorption (DVS) study.

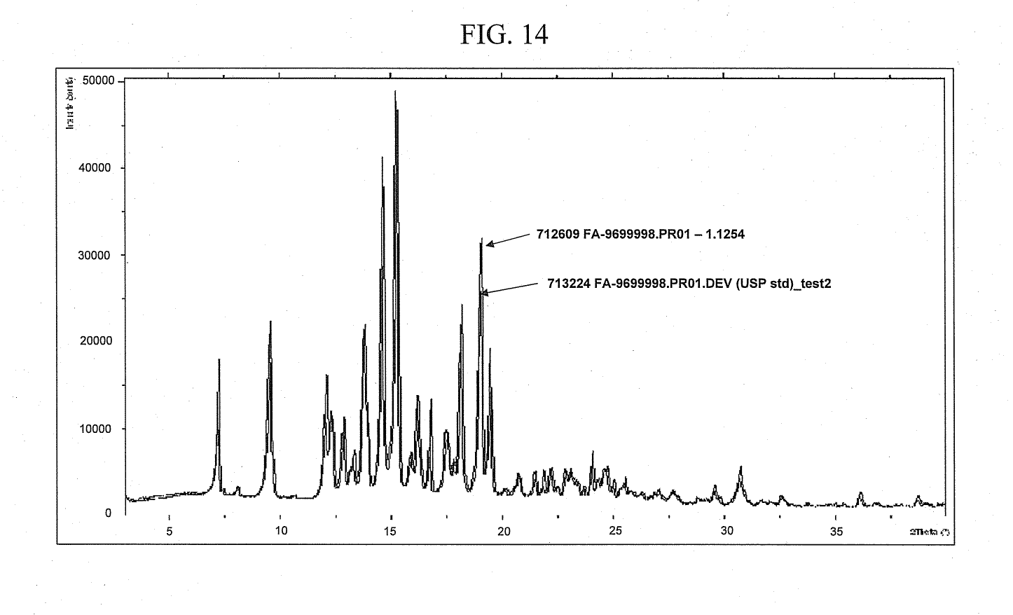

FIG. 14 depicts exemplary results of characterization of the bulk material 17-HPC using X-ray powder diffraction (XRPD).

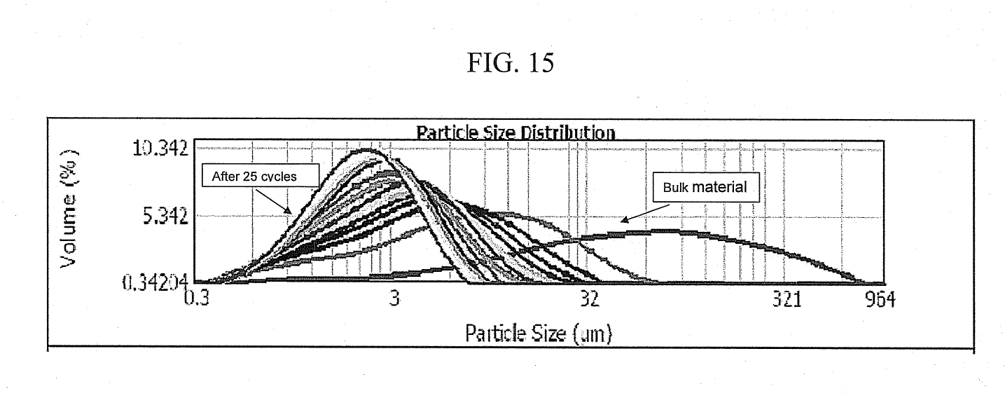

FIG. 15 depicts an exemplary API particle size distribution profile after particle size reduction from a suspension prepared with the surfactant Tween 80.

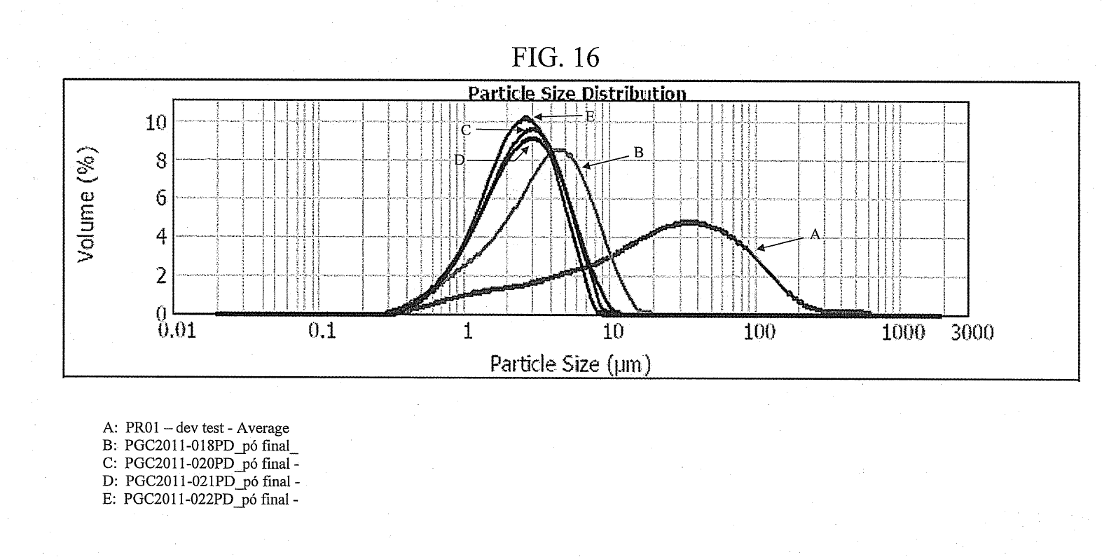

FIG. 16 depicts an exemplary API particle size distribution profile after particle size reduction from a 17-HPC dry powder after a spray-drying (SD) process.

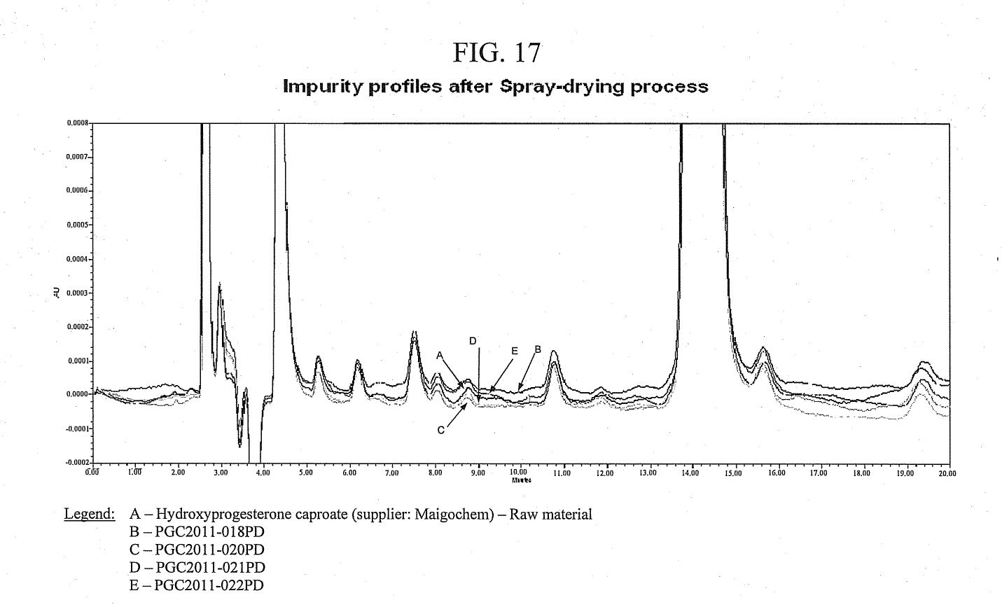

FIG. 17 depicts an exemplary impurity profile after particle size reduction of a 17-HPC dry powder obtained after a spray-drying (SD) process, as compared to the impurity profile of the starting bulk material.

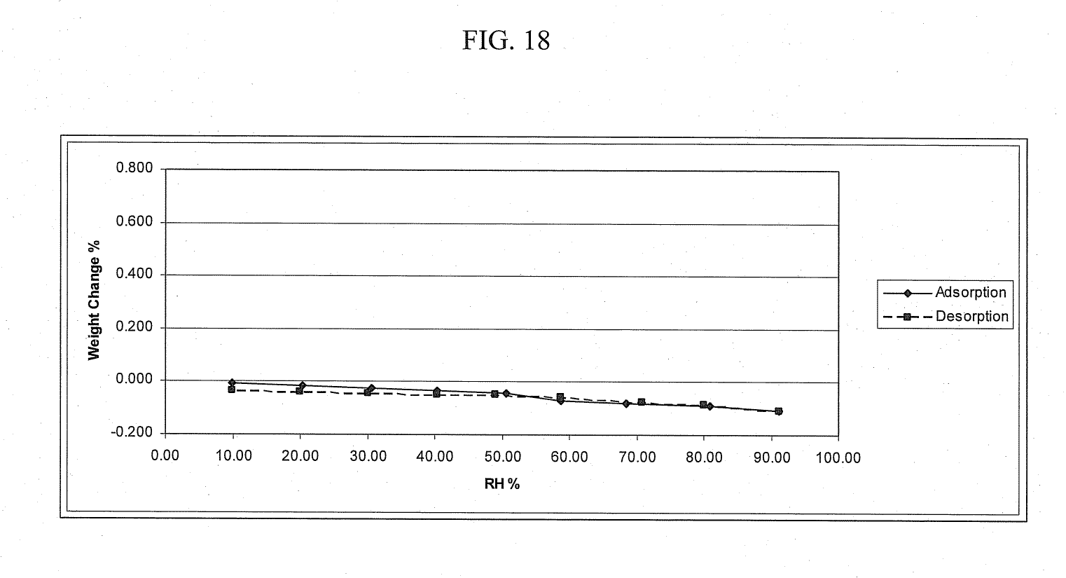

FIG. 18 depicts exemplary results after particle size reduction from a dynamic vapor sorption (DVS) analysis of a 17-HPC dry powder obtained after a spray-drying (SD) process.

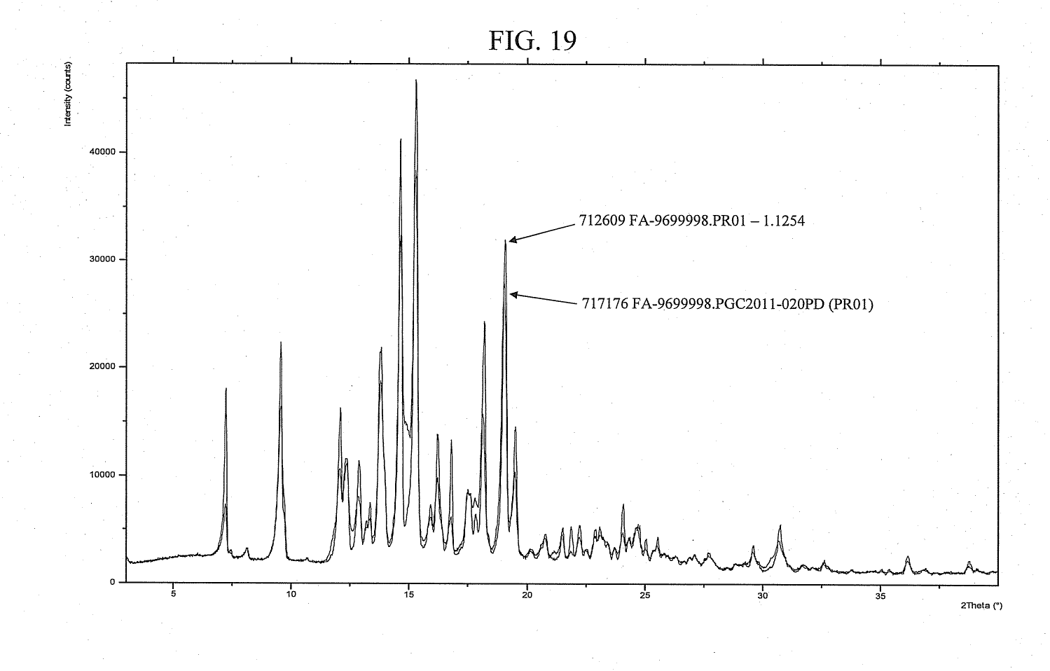

FIG. 19 depicts exemplary results after particle size reduction from an XRPD analysis of a 17-HPC dry powder obtained after a spray-drying (SD) process.

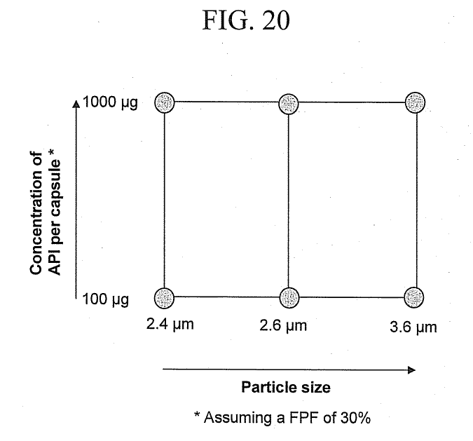

FIG. 20 depicts the concentration of API per capsule, relative to three different particle sizes, for exemplary dry powder blend formulations obtained after a spray-drying (SD) process with lactose.

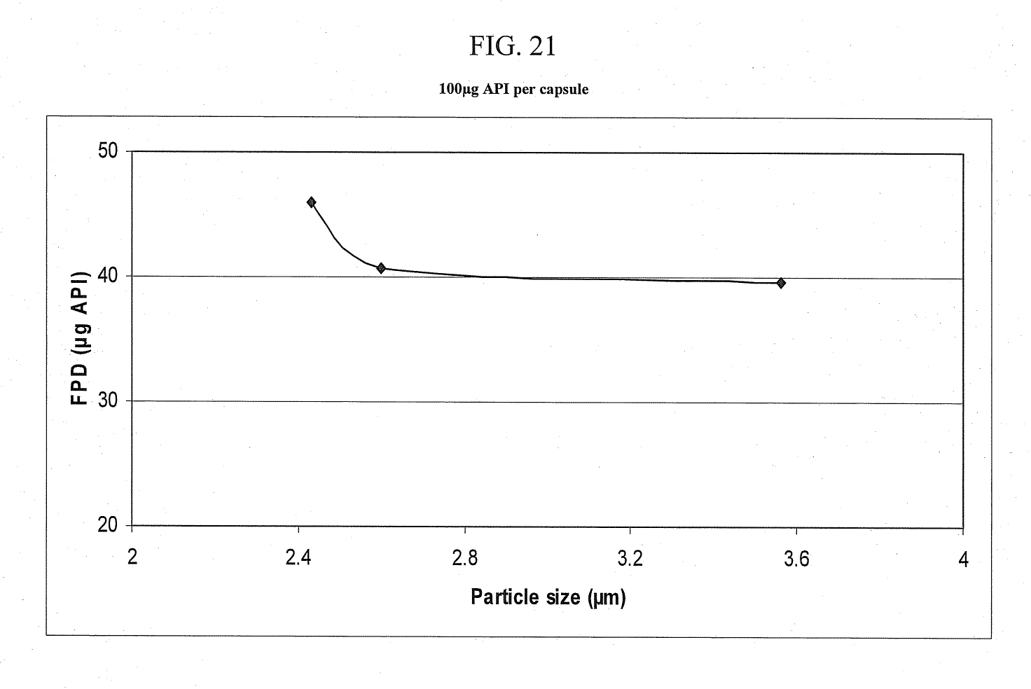

FIG. 21 depicts exemplary results showing a good correlation between particle size and fine-particle dose (FPD).

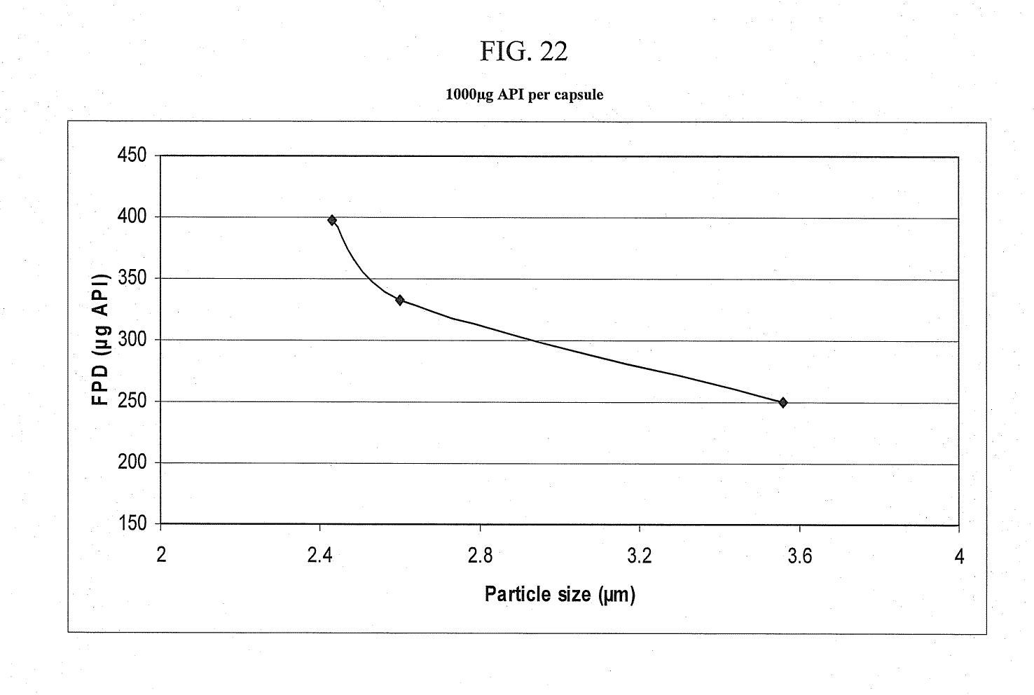

FIG. 22 depicts additional exemplary results showing a good correlation between particle size and fine-particle dose (FPD).

### DETAILED DESCRIPTION OF PREFERRED EMBODIMENTS

Described herein are preferred formulations including, for instance, inhalation formulations; methods; compositions; and kits according to the present invention which are suitable for restoring corticosteroid sensitivity, enhancing glucocorticoid sensitivity and/or reversing the glucocorticoid insensitivity in a subject experiencing corticosteroid dependence or corticoid resistance or unresponsiveness or intolerance to corticosteroids. Glucocorticoid insensitivity related conditions include, for instance, a range of immune-inflammatory disorders/diseases treated with steroids when the therapy fails to achieve disease control or is not effective or intolerant or dependent or resistant to corticosteroids, and combinations thereof. More specifically, the formulations, methods, compositions, and kits of the present invention are effective to achieve the glucocorticoid-sensitizer effects of steroid-sparing in corticosteroid-dependent patients, better responsiveness or tolerance to corticosteroids, achieving efficacy by using a lower dose of corticosteroid, preventing individuals at risk for developing corticosteroid refractory responses or resistance or exacerbations in response to antigen exposures, infections, exercise, or irritants, achieving optimal immune-functions, easier responses for the subject or patient when steroid administration is tapered or withdrawn, or in prolonged administration of corticosteroids, decreased risks for developing corticosteroid-related adverse events such as opportunistic infections and bone loss, and combinations thereof. More specifically, glucocorticoid insensitivity related conditions include various conditions/disorders associated with and/or attributed to glucocorticoid resistance, glucocorticoid refractory responses, corticosteroid dependence and corticosteroid intolerance. A primary risk factor in developing glucocorticoid insensitivity is oxidative stress.

Despite the serious consequences of glucocorticoid insensitivity, there has been limited success in the treatment of subjects who suffer from various conditions/disorders associated with and/or attributed to glucocorticoid resistance, glucocorticoid refractory responses, corticosteroid dependence and corticosteroid intolerance. Calcineurin inhibitors and methotrexate may be useful in a few selected conditions, but they have not proven to be effective in most glucocorticoid insensitivity related conditions. Moreover there are significant adverse events associated with their uses. Significant toxicity and side-effects have also hampered the drug development programs for p38 MAP-kinase inhibitors, selective inhibitors to block inhibition of NFκB kinase and Phosphodiesterase-4 inhibitors. The majority of glucocorticoid insensitivity disorders don't have any association with menstrual cycle, and most subjects suffering from glucocorticoid insensitivity disorders don't experience menstrual cycle-related exacerbation, including allergy to progesterone.

Advantageously in accordance with the present invention, the methods detailed herein are believed to significantly restore corticosteroid sensitivity in a subject preferably having no history of menstrual cycle-related exacerbation. The treatments and methods of the present invention have been surprisingly determined to reverse the glucocorticoid insensitivity and/or enhance glucocorticoid sensitivity in subjects suffering from at least one condition/disorder associated with and/or attributed to glucocorticoid resistance, glucocorticoid refractory responses, corticosteroid dependence and corticosteroid intolerance. Thus, the methods of the present invention are suitable for achieving the glucocorticoid-sensitizer effects of steroid-sparing in corticosteroid-dependent patients, better responsiveness or tolerance to corticosteroids, achieving efficacy by using a lower dose of corticosteroid, preventing individuals at risk for developing corticosteroid refractory responses or resistance or exacerbations in response to antigen exposures, infections, exercise, or irritants, achieving optimal immune-functions, easier responses when steroid administration is tapered or withdrawn, or in prolonged administration of corticosteroids, and decreased risks for developing corticosteroid-related adverse events such as opportunistic infections and bone loss. Preferably, the methods of the present invention are suitable for substantially negating the effect of at least one risk factor or underlying mechanism associated with glucocorticoid insensitivity. Thus, various embodiments of the present invention are directed to methods for reversing the glucocorticoid insensitivity in a subject preferably having no history of menstrual cycle-related exacerbation and suffering from one or more glucocorticoid insensitivity related conditions. In these and various other embodiments, the subject to be treated is either male or female, and of any age. Various other embodiments are directed to treating subjects that either have their first or already experienced repeated disease attacks without menstrual cycle-related exacerbation.

Although progesterone has anti-inflammatory properties trials of progesterone for inflammatory disorders such as rheumatoid arthritis have generally failed to demonstrate an effective and reproducible method for symptom control or better clinical outcomes. Subjects exhibiting a glucocorticoid-resistant or refractory response are a subset of the disease population, but a well-defined, “difficult to treat” subpopulation. For example, 20-30% of patients with severe and steroid-resistant Crohn's Disease will not respond to steroid therapy. One of the preferred objectives of the present invention is to use progesterone for treating a glucocorticoid-resistant or refractory condition demonstrated by steroid-sparing corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune-functions, easier responses for the subject when steroid administration is tapered or withdrawn, or in or after prolonged administration of corticosteroids, decreased risks for developing corticosteroid-related adverse events such as opportunistic infections and bone loss, and combinations thereof. Furthermore, MAPK activation is a critical event that leads to corticosteroid-insensitivity. It has been reported that progesterone increases MAPK activity (Ruzycky A L. Effects of 17 beta-estradiol and progesterone on mitogen-activated protein kinase expression and activity in rat uterine smooth muscle. Eur J. Pharmacol. 1996 Apr. 11; 300(3):247-54). Without being bound to a particular theory, it is currently believed that a skilled artisan would expect that MAPK activation induces a loss of GR nuclear translocation and function, leading to the development of corticosteroid-insensitivity related conditions. Again, without being bound to a particular theory, it is further currently believed that a skilled artisan would expect that the increased MAPK by progesterone would aggravate corticosteroid-insensitivity. However, in accordance with the present invention it has been surprisingly and unexpectedly discovered that the molecular effects of increased MAPK by progesterone do not interfere with the effectiveness of progesterone in treating a glucocorticoid-resistant or refractory condition. It has further been surprisingly and unexpectedly discovered that administration of progesterone such as 17alpha-hydroxyprogesterone caproate (17-HPC) to a subject with a glucocorticoid-resistant or refractory or corticosteroid-dependent condition achieves the glucocorticoid-sensitizer effects such as steroid-sparing. Thus, in accordance with the present invention, it is currently believed that administration of progesterone (e.g., 17-HPC) can significantly restore corticosteroid sensitivity, enhance glucocorticoid sensitivity and/or reverse glucocorticoid insensitivity.

In comparison to the use of progesterone for its anti-inflammatory effects, the present discovery has surprisingly identified a new function of progestogen and its uses, i.e., reversing corticosteroid insensitivity, and clearly identifies a well-defined patient population who would benefit from the treatment, i.e., patients exhibiting corticosteroid resistance, corticosteroid dependence, corticosteroid refractory responses, and/or corticosteroid intolerance. Since most glucocorticoid insensitivity related conditions occur in subjects that do not have a history of menstrual cycle-related exacerbation, the present discovery also represents a significant advance in the art.

### DEFINITIONS

As noted, glucocorticoids remain the first-line treatment for a range of immune/inflammatory and allergic diseases. However, 30% of patients fail to achieve disease control at tolerable systemic doses and continue to have an increased immune response with poor clinical outcomes. The glucocorticoid insensitivity is an important factor in the pathogenesis and prognosis of many diseases. It presents considerable management problems and cost burdens to health services. As used herein, the term “glucocorticoid insensitivity” is intended to include, but is not limited to, corticosteroid resistance, corticosteroid dependence, corticosteroid refractory responses, corticosteroid intolerance, and other types of corticosteroid ineffectiveness. It has been recognized that several distinct molecular mechanisms contribute to decreased anti-inflammatory effects of glucocorticoids. Different inflammatory diseases may share similar molecular mechanisms, and a single disease may have a heterogeneity of mechanisms.

Corticosteroid resistance to the anti-inflammatory effects of corticosteroids is defined as no clinical improvement after treatment with high-dose glucocorticoid.

Corticosteroid dependence is defined as a condition that initially responds to corticosteroids but relapses quickly upon drug withdrawal or dose tapering.

Corticosteroid refractory response is defined as a condition that does not respond to an adequate induction dose of corticosteroids. It includes relatively or totally refractory responses to glucocorticoid therapy, and often needs to be controlled by add-on treatment.

Other types of corticosteroid ineffectiveness includes need for a very high dose treatment, “difficult to treat” and “do not respond well” or severe cases, and impaired in vitro and in vivo responsiveness.

Corticosteroid intolerance is defined as toxicity of the therapy and/or risks for developing corticosteroid-related adverse events such as opportunistic infections and bone loss.

Glucocorticoid sensitizer is defined as a pharmaceutical agent and product that has a function in restoring corticosteroid sensitivity, enhancing glucocorticoid sensitivity, reversing the glucocorticoid insensitivity, and protecting against loss of glucocorticoid sensitivity, and used for treating, preventing, or ameliorating one or more of the symptoms of diseases or disorders associated with glucocorticoid insensitivity (e.g., corticosteroid dependent or corticoid resistant or unresponsive or intolerant to corticosteroids). Therapeutic effects of the use of a glucocorticoid sensitizer include any, but are not limited to, steroid-sparing in corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune functions, easier responses for the subject or patient when steroid administration is tapered or withdrawn, or after prolonged administration of corticosteroids, decreased risks for developing corticosteroid-related adverse events such as opportunistic infections, bone loss, pathologic fracture, diabetes, cataract, and combinations thereof.

As used herein, the terms “treating” or “treatment” encompass either or both responsive and prophylactic measures, e.g., designed to inhibit or delay the onset of the disease or disorder, achieve a full or partial reduction of the symptoms or disease state, and/or to alleviate, ameliorate, lessen, or cure the disease or disorder and/or its symptoms. Treatment also encompasses any pharmaceutical use of the compositions of the present invention, such as use for treating a glucocorticoid insensitivity related disease or disorder or condition. Amelioration of the symptoms of a particular disorder by administration of a particular compound or pharmaceutical composition of the present invention refers to any lessening, whether permanent or temporary, lasting or transient that can be attributed to or associated with administration of the composition.

Subject is defined herein as an animal, typically a mammal, including human. As used herein, the term “patient” includes human and animal subjects.

As used herein throughout the description, it is to be understood that the phrase “dry powder” is intended to refer to substantially dry powder, i.e., a powder in which substantially all the moisture content is removed; and in preferred embodiments, the phrase refers to powder that is essentially devoid of any measurable moisture content.

Preferred Formulations and Routes of Delivery

In practicing the methods of the present invention, effective amounts of the progesterone compounds or compositions containing therapeutically effective concentrations of the compounds, may be formulated in any suitable manner to achieve therapeutically effective results in a subject.

Inhalation Formulations

A particularly preferred route of delivery for administering effective amounts of the progesterone compounds or compositions containing therapeutically effective concentrations of the compounds is via an inhalation route of administration. When an inhalation route of administration is used, delivery may preferably be, for example, via an aerosol spray or powder mixture in a pressurized pack or a nebulizer or in an inhaler.

Inhalation formulations may be used for the treatment of glucocorticoid-insensitivity related diseases or disorders, or conditions, including, but not limited to, glucocorticoid resistant conditions (e.g., cigarette smoking-related lung diseases such as chronic obstructive pulmonary disease, Asthma, Chronic Bronchitis, Emphysema, Influenza, Acute Non-Influenzal Respiratory Disease, Pneumonia, Tuberculosis, lung cancer, interstitial lung disease, including respiratory bronchiolitis, desquamative interstitial pneumonitis, pulmonary Langerhans cell histiocytosis and combined pulmonary fibrosis and emphysema (CPFE), acute respiratory distress syndrome, interstitial pulmonary fibrosis, and cystic fibrosis; glucocorticoid refractory conditions (e.g., refractory ulcerative colitis, children with severe Crohn disease, corticosteroid refractory asthma, desquamative interstitial pneumonia refractory to corticosteroid, refractory inflammatory myopathies, refractory myasthenia gravis, refractory pemphigus vulgaris, methotrexate-refractory RA patients, refractory nephrotic syndrome, refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, critically ill patients with sepsis or acute respiratory distress syndrome (ARDS) and relative adrenal insufficiency); glucocorticoid dependent conditions (e.g., rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, corticosteroid dependent systemic lupus erythematosus, corticosteroid dependent multiple sclerosis, symptomatic corticosteroid dependent asthma, primary Sjogren's syndrome, systemic vasculitis, polymyositis, organ transplants, and graft-versus-host disease); and other inflammatory diseases, autoimmune diseases, hyperproliferative diseases, and other such disease when glucocorticoid-insensitivity is implicated. Exemplary of these diseases are lupus, osteoarthritis, rhinosinusitis, polyarteritis nodosa, Wegener's granulomatosis, giant cell arteritis, allergic rhinitis, urticaria, hereditary angioedema, tendonitis, bursitis, autoimmune chronic active hepatitis, cirrhosis, transplant rejection, psoriasis, dermatitis, malignancies (e.g., leukemia, myelomas, lymphomas), acute adrenal insufficiency, rheumatic fever, granulomatous disease, immune proliferation/apotosis, hypothalamic-pituitary-adrenal (HPA) axis suppression and regulation, hypercortisolemia, modulation of the Th1/Th2 cytokine balance, chronic kidney disease, spinal cord injury, cerebral edema, thrombocytopenia, Little's syndrome, Addison's disease, autoimmune hemolytic anemia, uveitis, pemphigus vulgaris, nasal polyps, sepsis, infections (e.g., bacterial, viral, rickettsial, parasitic), type II diabetes, obesity, metabolic syndrome, depression, schizophrenia, mood disorders, Cushing's syndrome, anxiety, sleep disorders, memory and learning enhancement, or glucocorticoid-induced glaucoma, atopic dermatitis, drug hypersensitivity reactions, serum sickness, bullous dermatitis herpetiformis, contact dermatitis, exfoliative erythroderma, mycosis fungoides, pemphigus, nonsuppurative thyroiditis, sympathetic ophthalmia, uveitis and ocular inflammatory conditions unresponsive to topical steroids, allergic bronchopulmonary aspergillosis, fulminating or disseminated pulmonary tuberculosis when used concurrently with appropriate chemotherapy, hypersensitivity pneumonitis, idiopathic bronchiolitis obliterans with organizing pneumonia, idiopathic eosinophilic pneumonias, idiopathic pulmonary fibrosis, pneumocystis carinii pneumonia (PCP) associated with hypoxemia occurring in an HIV(+) individual who is also under treatment with appropriate anti-PCP antibiotics, a diuresis or remission of proteinuria in nephritic syndrome, without uremia, of the idiopathic type or that due to lupus erythematosus, ankylosing spondylitis, polymyalgia rheumatic, psoriatic arthritis, relapsing polychondritis, trichinosis with neurologic or myocardial involvement, and tuberculous meningitis.

Exemplary 17-HPC Powder Formulations

As described herein, when an inhalation route of administration is used, delivery may preferably be accomplished, for example, via an aerosol spray or powder mixture in a pressurized pack or a nebulizer or in an inhaler.

It is preferred that pharmaceutically acceptable compositions for inhalation delivery include dry powders comprising an active ingredient (for instance, 17-HPC) present in a dry bulking powder suitable for dry powder inhalation or suspensions suitable for nebulization, and aerosol propellants suitable for use in a metered dose inhaler.

One particularly preferred exemplary formulation is a 17-HPC powder formulation for dry powder inhalation. Moreover, it is preferred that the 17-HPC powder formulation for administration by inhalation comprises the 17-HPC active substance and a pharmaceutically acceptable excipient (e.g., lactose). It is also preferred, according to one embodiment of the present invention, that the composition has the form of a physical mixture (for instance, a powder blend) and comprises from about five (5) to about fifty (50) weight percent of the excipient, and wherein the active substance (17-HPC) has a particle size distribution profile of from about one nanometer to about ten (10) microns (μm), and wherein the excipient has a particle size distribution of from about fifteen (15) to about five-hundred (500) microns. It is to be understood, in accordance with other embodiments of the present invention, that the compositions of the present invention can alternatively have other particle size distribution profiles as needed or desired, wherein said compositions are suitable and effective for administration to a subject, for instance, administration by inhalation.

Pulmonary local delivery of 17-HPC and progesterone to a subject (for instance, a human) is preferably accomplished by inhalation through the mouth. Surprisingly, it has been found in accordance with the present invention that respiratory (i.e., inhalation or pulmonary) delivery of the 17-HPC active ingredient is safe, in contrary to the previous conventional belief that 17-HPC and progesterone are harmful if they are inhaled. This surprising and unexpected finding, in accordance with the present invention, represents a significant discovery.

Moreover, another surprising and unexpected finding, in accordance with the present invention, is that particle size reduction of 17-HPC to a particle size distribution that ranges from about one nanometer to about ten (10) microns is optimal for a therapeutically effective powder composition (e.g., powder blend). According to certain preferred aspects of the invention, particle size reduction of 17-HPC, for instance, preferably substantially hydrophobic 17-HPC, can be achieved by milling in water, either with a surfactant or without a surfactant, wherein the particle size reduction of 17-HPC is achieved without changing its basic crystalline structure and without generating any measurable additional impurity or impurities.

In addition, it has also been surprisingly discovered, in accordance with the present invention, that one or more pharmaceutically acceptable surfactants may be used in achieving optimal particle size reduction, i.e., the reduction in API particle size, for instance, 17-HPC particle size reduction. One preferred surfactant is Tween 80, which can preferably be used at a concentration of from about five (5) to about fifteen (15) percent. In addition to Tween 80, other examples of pharmaceutically acceptable surfactants that may be used in accordance with the present invention include, but are not limited to, e.g., monoglycerides, di-glycerides, polysorbate 60, sorbitol-fatty acid esters, and glycerol-lactic acid esters. Additional examples of surfactants include, but are not limited to, polyoxyethylene (hereinafter abbreviated as POE-branched alkyl ethers such as POE-octyldodecyl alcohol and POE-2-decyltetradecyl alcohol, POE-alkyl ethers such as POE-oleyl alcohol ether and POE-cetyl alcohol ether, sorbitan esters such as sorbitan monooleate, sorbitan monoisostearate and sorbitan monolaurate, POE-sorbitan esters such as POE-sorbitan monooleate, POE-sorbitan monoisostearate and POE-sorbitan monolaurate, fatty acid esters of glycerol such as glyceryl monooleate, glyceryl monostearate and glyceryl monomyristate, POE-fatty acid esters of glycerol such as POE-glyceryl monooleate, POE-glyceryl monostearate and POE-glyceryl monomyristate, POE-dihydrocholesterol ester, POE-hardened castor oil, POE-hardened castor oil fatty acid esters such as POE-hardened castor oil isostearate, POE-alkylaryl ethers such as POE-octylphenol ether, glycerol esters such as glycerol monoisostearate and glycerol monomyristate, POE-glycerol ethers such as POE-glycerol monoisostearate and POE-glycerol monomyristate, polyglycerol fatty acid esters such as diglyceryl monostearate, decaglyceryl decastearate, decaglyceryl decaisostearate and diglyceryl diisostearate and other nonionic surfactants; potassium salts, sodium salts, diethanolamine salts, triethanolamine salts, amino acid salts and other salts of higher fatty acids such as myristic acid, stearic acid, palmitic acid, behenic acid, isostearic acid and oleic acid, the above alkali salts of ether carboxylic acids, salts of N-acylamino acids, N-acylsalconates, higher alkylsulfonates and other anionic surfactants; alkylamine salts, polyamine, aminoalcohol fatty acids, organic silicone resin, alkyl quaternary ammonium salts and other cationic surfactants; and lecithin, betaine derivatives and other amphoteric surfactants. It is to be understood that other surfactants may also be used.

In preferred embodiments of the present invention, 17-HPC compositions include dry powders that comprise the 17-HPC present in a dry bulking powder suitable for dry powder inhalation; or suspensions comprising 17-HPC suitable for nebulization, or alternatively, aerosol propellant formulations suitable for use with a metered dose inhaler. It is preferred to achieve a fine-particle dose (FPD) of 17-HPC in the range of approximately about fifteen (15) to about six-hundred (600) micrograms (μg), wherein FPD is defined as the dose of the aerosolized drug particles with an aerodynamic diameter less than about five (5) microns.

In preferred embodiments of the present invention, and referring to FIGS. 21 and 22, the composition for inhalation delivery exhibits a desired correlation such that a relatively small particle size distribution (for instance, less than about 3.6 microns) correlates with a desired fine-particle dose (FPD) of 17-HPC, e.g., FPD in the range of between about fifteen (15) to about six-hundred (600) micrograms (μg). Moreover, it is preferred that the compositions of the present invention are characterized by a blend homogeneity having a relative standard deviation (RSD) less than about five percent, and it is also preferred that the compositions for inhalation delivery have a fine particle fraction (FPF) of about thirty percent or greater. It is also to be understood that blend homogeneity can be determined by any suitable method, for instance, by high-performance liquid chromatography (HPLC).

Exemplary Techniques for Bulk Material Characterization

For purposes of characterizing bulk material used, for instance, in a powder formulation, e.g., a 17-HPC powder formulation, any suitable technique or method can be used in accordance with the present invention for characterizing the bulk material. Characterization of the bulk material can be performed, for instance, using bulk powder density analyzers; X-Ray Powder Diffraction (XRPD); water vapor sorption; or dynamic vapor sorption (DVS) techniques.

XRPD is an established and very reliable technique for determining crystalline structure.

Dynamic vapor sorption (DVS) is a gravimetric technique that measures how quickly and how much of a solvent is absorbed by a sample, such as a dry powder absorbing water. DVS accomplishes this by varying the vapor concentration surrounding the sample and measuring the change in mass which this produces.

Exemplary Techniques for Characterizing Particle Size and Distribution

In accordance with the present invention, any suitable technique or method can be used for characterizing particle size and particle size distribution, for instance, the particle size distribution of an active pharmaceutical ingredient (API) in a powder formulation. Exemplary methods include, for instance, the use of one or more of surface area analysis, pore size analysis, continuous-imaging particle analysis, powder characterization, diffraction laser particle size analysis; pattern recognition techniques; and imaging particle analysis, just to name a few examples. Imaging particle analysis systems, for instance, with laser-scatter triggering, can accurately calculate concentrations of particles in relatively concentrated samples. For sparse samples, methods using a laser-scatter trigger signal can be used to image and measure particles in a sparse sample.

Pattern recognition techniques can also be used to identify and differentiate different particle types contained in a heterogeneous solution. Pattern recognition techniques may involve, for instance, imaging microscopic particles in real-time as they flow in a solution, segregating each individual particle as a separate image, and then applying pattern recognition techniques to differentiate the individual particle types.

Laser diffraction instrumentation may also be used for characterizing particle size and particle size distribution. Particle size and particle size distribution can be determined from a detected diffraction pattern using an appropriate scattering model.

In determining and characterizing particle size, e.g., particle size of an API (active pharmaceutical ingredient), the particle size parameters Dv(10), Dv(50) and Dv(90) may be used. Particle size measurements are preferably expressed in terms of Dv(10), Dv(50), and Dv(90), wherein Dv(10) refers to the particle size below which 10% of the volume of material exists; Dv(50) refers to the particle size below which 50% of the volume of material exists; and Dv(90) refers to the particle size below which 90% of the volume of material exists.

Other Routes of Delivery

Other routes of delivery may be used for administering effective amounts of the progesterone compounds or compositions containing therapeutically effective concentrations of the compounds. For instance, the present invention also contemplates formulations for systemic delivery, including for instance parenteral, oral, or intravenous delivery, or for local or topical application, for the treatment of glucocorticoid-insensitivity related diseases or disorders, or conditions, including, but not limited to, glucocorticoid resistant conditions (e.g., glucocorticoid resistant asthma, refractory rheumatoid arthritis, refractory inflammatory bowel disease, chronic obstructive pulmonary disease and acute respiratory distress syndrome, interstitial pulmonary fibrosis, and cystic fibrosis); glucocorticoid refractory conditions (e.g., refractory ulcerative colitis, children with severe Crohn disease, corticosteroid refractory asthma, desquamative interstitial pneumonia refractory to corticosteroid, refractory inflammatory myopathies, refractory myasthenia gravis, refractory pemphigus vulgaris, methotrexate-refractory RA patients, refractory nephrotic syndrome, refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, critically ill patients with sepsis or acute respiratory distress syndrome (ARDS) and relative adrenal insufficiency); glucocorticoid dependent conditions (e.g., rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, corticosteroid dependent systemic lupus erythematosus, corticosteroid dependent multiple sclerosis, symptomatic corticosteroid dependent asthma, primary Sjogren's syndrome, systemic vasculitis, polymyositis, organ transplants, and graft-versus-host disease); and other inflammatory diseases, autoimmune diseases, hyperproliferative diseases, and other such disease when glucocorticoid-insensitivity is implicated. Exemplary of these diseases are lupus, osteoarthritis, rhinosinusitis, polyarteritis nodosa, Wegener's granulomatosis, giant cell arteritis, allergic rhinitis, urticaria, hereditary angioedema, tendonitis, bursitis, autoimmune chronic active hepatitis, cirrhosis, transplant rejection, psoriasis, dermatitis, malignancies (e.g., leukemia, myelomas, lymphomas), acute adrenal insufficiency, rheumatic fever, granulomatous disease, immune proliferation/apotosis, hypothalamic-pituitary-adrenal (HPA) axis suppression and regulation, hypercortisolemia, modulation of the Th1/Th2 cytokine balance, chronic kidney disease, spinal cord injury, cerebral edema, thrombocytopenia, Little's syndrome, Addison's disease, autoimmune hemolytic anemia, uveitis, pemphigus vulgaris, nasal polyps, sepsis, infections (e.g., bacterial, viral, rickettsial, parasitic), type II diabetes, obesity, metabolic syndrome, depression, schizophrenia, mood disorders, Cushing's syndrome, anxiety, sleep disorders, memory and learning enhancement, or glucocorticoid-induced glaucoma, atopic dermatitis, drug hypersensitivity reactions, serum sickness, bullous dermatitis herpetiformis, contact dermatitis, exfoliative erythroderma, mycosis fungoides, pemphigus, nonsuppurative thyroiditis, sympathetic ophthalmia, uveitis and ocular inflammatory conditions unresponsive to topical steroids, allergic bronchopulmonary aspergillosis, fulminating or disseminated pulmonary tuberculosis when used concurrently with appropriate chemotherapy, hypersensitivity pneumonitis, idiopathic bronchiolitis obliterans with organizing pneumonia, idiopathic eosinophilic pneumonias, idiopathic pulmonary fibrosis, pneumocystis carinii pneumonia (PCP) associated with hypoxemia occurring in an HIV(+) individual who is also under treatment with appropriate anti-PCP antibiotics, a diuresis or remission of proteinuria in nephritic syndrome, without uremia, of the idiopathic type or that due to lupus erythematosus, ankylosing spondylitis, polymyalgia rheumatic, psoriatic arthritis, relapsing polychondritis, trichinosis with neurologic or myocardial involvement, and tuberculous meningitis.

Generally, in accordance with the present invention, the methods described herein for the treatment of glucocorticoid-insensitivity related diseases or disorders, or conditions comprise administering a pharmaceutical composition comprising a steroid hormone. Typically, the lipophilic gonadal steroid hormone is a progestogen. The progestogen may be a naturally occurring progestogen or a synthetic progestogen (i.e., a progestin). Progestogens that can be used in accordance with the present invention are grouped into the following categories: progesterone, retroprogesterone, progesterone derivative, 17α-OH progesterone derivatives (both pregnanes and norpregnanes), 19-norprogesterone derivatives, 19-nortestosterone derivatives (both estranges and gonanes), and spironolactone derivatives. Generally, the progestogen for use in accordance with the present invention is selected from the group consisting of progesterons and their derivatives or active metabolites. Specific examples of progestogens that may be used in the methods and kits of the present invention include, but are not limited to, 17alpha-HPC hydroxyprogesterone, natural progesterone, dydrogesterone, medrogestone, medroxyprogesterone, megestrol acetate, chlormadinone acetate, cyproterone acetate, gestonorone caproate, nomegestrol acetate, demegestone, promegestone, nestorone, trimegestone, norethisterone acetate, norethisterone, lynestrenol, ethynodiol diacetate, norgestrel, levonorgestrel, desogestrel, etonogestrel (3-ketodesogestrel), gestodene, norgestimate, norelgestromin (17-deacetyl norgestimate), dienogest, drospirenone, norethynodrel, norgestrel, desogestrel, etonogestrel, 19-nortestosterone, dienogest, norethynodrel, cyproterone acetate, tibolone, 19-norprogesterone, and drospirenone.

Other agents that can be used in accordance with the methods and kits of the present invention include, for example, any pharmaceutically-acceptable progestogen derivatives, i.e., derivatives of 17alpha-HPC hydroxyprogesterone, natural progesterone, dydrogesterone, medrogestone, medroxyprogesterone, megestrol, chlormadinone, cyproterone, gestonorone caproate, nomegestrol acetate, demegestone, promegestone, nestorone, trimegestone, norethisterone, norethisterone, lynestrenol, ethynodiol diacetate, norgestrel, levonorgestrel, desogestrel, etonogestrel (3-ketodesogestrel), gestodene, norgestimate, norelgestromin (17-deacetyl norgestimate), dienogest, drospirenone, norethynodrel, norgestrel, desogestrel, etonogestrel, 19-nortestosterone, dienogest, norethynodrel, cyproterone, tibolone, 19-norprogesterone, and drospirenone. Each progestogen can be derivatized as the corresponding salts, esters, enol ethers or esters, acids, bases, solvates, hydrates or prodrugs prior to formulation, as described herein. Representative pharmaceutically-acceptable salts include, but are not limited to, amine salts, such as but not limited to, chloroprocaine, choline, ammonia; diethanolamine and other hydroxyalkylamines, ethylenediamine, Nmethylglucamine, procaine, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. For example, the organic acid of acetates is often used such as megestrol acetate, chlormadinone acetate, cyproterone acetate, gestonorone caproate, nomegestrol acetate, and cyproterone acetate.

Additional representative agents that can be used in accordance with the methods and kits of the present invention include, for example, any progestogen active metabolite including, but not limited to, active metabolites of 17alpha-HPC hydroxyprogesterone, natural progesterone, dydrogesterone, medrogestone, medroxyprogesterone, megestrol acetate, chlormadinone acetate, cyproterone acetate, gestonorone caproate, nomegestrol acetate, demegestone, promegestone, nestorone, trimegestone, norethisterone acetate, norethisterone, lynestrenol, ethynodiol diacetate, norgestrel, levonorgestrel, desogestrel, etonogestrel (3-keto-desogestrel), gestodene, norgestimate, norelgestromin (17-deacetyl norgestimate), dienogest, drospirenone, norethynodrel, norgestrel, desogestrel, etonogestrel, 19-nortestosterone, dienogest, norethynodrel, cyproterone acetate, tibolone, 19-norprogesterone, and drospirenone. For example, active metabolites of progesterone include allopregnanolone and 5alphapregnan-3,20-dione the active metabolite. Active metabolites of 17-HPC include M13 monohydroxy-; M12, monohydroxy-; M19, monohydroxy-; M7, dihydroxy-; and M16, monohydroxy-.

In various embodiments, another group of steroid hormone, glucocorticoids, for use in accordance with the present invention is preferably selected from the group consisting of naturally produced steroid hormones, or synthetic compounds, that inhibit the process of inflammation. Specific examples of glucocorticoids include, but are not limited to, hydrocortisone (cortisol), cortisone acetate, dexamethasone (hereinafter, “Dexamethasone”), prednisone, prednisolone, methylprednisolone, betamethasone, triamcinolone, beclometasone, Paramethasone, fluticasone, fludrocortisone acetate, deoxycorticosterone acetate (DOCA), Fluprednisolone, fluticasone propionate, budesonide, beclomethasone dipropionate, flunisolide and triamcinolone acetonide.

In practicing the methods of the present invention, effective amounts of the compounds or compositions containing therapeutically effective concentrations of the compounds, are preferably formulated for systemic delivery, including parenteral, oral, or intravenous delivery, or for local or topical application. For example, the pharmaceutical composition may be administered by subcutaneous, intravenous, intraperitoneal, intraarterial or intramuscular injection; rectally; by transdermally delivery; intravaginal delivery; or buccally; or by oral delivery. When administered by subcutaneous or intramuscular injection, the steroid hormone is suitably formulated as a depot formulation to allow for sustained release of the steroid hormone over an extended period of time. When administered by topical administration, including intravaginal delivery, delivery may suitably be, for example, via a solution, suspension, emulsions or the like and are preferably formulated as creams, gels, ointments, emulsions, solutions, elixirs, lotions, suspensions, tinctures, pastes, foams, aerosols, irrigations, sprays, suppositories, bandages, dermal patches or any other formulations suitable for the route.

With respect to the frequency of administration, any frequency which achieves the desired result (i.e., steroid-sparing in corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune-functions, easier responses for the subject when steroid administration is tapered or withdrawn, or after prolonged administration of corticosteroids, decreased risks for developing corticosteroid-related adverse events such as opportunistic infections and bone loss, and combinations thereof, may be used. The frequency of administration will preferably be determined, at least in part, by the steroid hormone(s) and/or dosage form selected. In various embodiments, the pharmaceutical composition is preferably administered at an interval exceeding daily or once per week. For example, the pharmaceutical composition may be administered once every other week, once monthly, once every two months, or once every three months. In various other embodiments, the pharmaceutical composition is administered once weekly, or at an interval of less than one week (e.g., daily or every other day). For example, when the steroid hormone is 17alpha-hydroxyprogesterone caproate (17-HPC), administration may suitably be via daily, once-weekly or once every two-week, or once-monthly or once every 3-month injections. Those skilled in the art will understand that the route of administration and frequency of administration for the pharmaceutical compositions used in the methods and kits of the present invention will depend on a variety of factors including, for example, the particular steroid hormone(s) used, the formulation in which it is delivered, the tissue being treated, the age and gender of the individual treated, in vivo or in vitro test data, and the professional judgment of the particular patient's needs. The dosing frequency ranges set forth herein are exemplary only and are not intended to limit the scope or practice of formulations provided herein.

The skilled artisan will also appreciate that appropriate dosing of the steroid hormone will depend on the steroid hormone(s) selected, the route of administration and dosage form, the frequency of administration, the disease(s) to be treated, the metabolic stability and length of action of that compound, the species, age, body weight, general health, and diet of the subject, rate of excretion, drug combination, and severity of the particular condition. The effective amount of a steroid hormone provided herein can be determined by one of ordinary skill in the art, and includes exemplary dosage amounts for a mammal of from about 0.001 to 100 mg/kg of body weight of active compound given orally per day. For example, to achieve the endometrium and antigonadotropic effects (i.e., dose for ovulation inhibition), 0.15 mg/day p.o. for Levonorgestrel or Desogestrel is preferably desired while the required amount is much higher, 5-10 mg/day for Medroxyprogesterone acetate or 200-300 mg/day for Progesterone.

The skilled artisan will also appreciate that appropriate dosing of the steroid hormone will depend on gender as progestogen is the sex hormone. Progesterone is primarily secreted by the granulosa cells and the corpus luteum in the ovary. During pregnancy, a major source of progesterone also comes from the placenta. Males produce progesterone in the adrenal gland and testes, as this is a precursor of testosterone. In women, progesterone levels are relatively low during the preovulatory phase of the menstrual cycle, rise after ovulation, and are elevated during the luteal phase. Progesterone levels tend to be <2 ng/ml prior to ovulation, and >5 ng/ml after ovulation. If pregnancy occurs, progesterone levels are initially maintained at luteal levels. With the onset of the luteal-placental shift in progesterone support of the pregnancy, levels start to rise further and may reach 100-200 ng/ml at team. The reference range for progesterone levels in adult men is 0.13-0.97 ng/ml. Adult males have levels similar to those in women during the follicular phase of the menstrual cycle as well as the level in postmenopausal women. Clearly, women regularly experience a 17-fold change in serum progesterone concentration during the menstrual cycle, or more than 100-fold increase in pregnancy. Thus, tolerance or maximum dose or minimal effective dose of progestogen treatment would be higher in women than in males. For example, when the steroid hormone is 17alpha-hydroxyprogesterone caproate (17-HPC) and a common dosage used is 150-500 mg weekly injection for its uses in women-health related indications. Given some important effects of progesteron on restoring corticosteroid sensitivity are assumed to be mediated non-genomically through different molecular biological modes of action (i.e., functions not related to progestational activity), this may result in some pharmacodynamic variability. A much lower or higher dose of progesterone (e.g. 17-HPC) may be selected as well as a different dosage level for male subjects. The dosing ranges set forth herein are exemplary only and are not intended to limit the scope or practice of formulations provided herein.

Exemplary Dosage Forms and Dosage Administrations

Preferably, the pharmaceutical compositions of the present invention contain: i) a physiologically acceptable carrier, diluent, or excipient, or a combination thereof; and ii) one or more steroid hormone(s) as described herein. The compositions can be formulated for single dosage administration or for multiple dosages. Dosage forms or compositions containing steroid hormone(s), for instance, in the range of about 0.005% to about 100%, with the balance of the dosage form or composition made up of one or morenon-toxic carriers and/or pharmaceutically acceptable excipients, can be prepared.

For example, an exemplary pharmaceutical composition in accordance with the present invention may contain one or more diluents, one or more carriers, one or more binders, one or more coatings, one or more lubricants, one or more solvents, one or more buffers, one or more preservatives, one or more flavoring agents, one or more dyes, and/or one or more absorption enhancers, and/or one or more biodegradable polymers.

The particular excipient(s) included in the pharmaceutical composition will depend on the particular steroid hormone(s) and dosage form selected, and the skilled artisan will be able to readily select appropriate excipients once the steroid hormone(s) and the dosage form therefore have been chosen.

For example, for oral administration, a pharmaceutically acceptable non-toxic composition in accordance with the present invention can preferably be formed by the incorporation of any of one or more normally employed excipients, such as, for example, pharmaceutical grades of mannitol, lactose, starch, magnesium stearate, talcum, cellulose derivatives, sodium crosscarmellose, glucose, sucrose, magnesium carbonate or sodium saccharin. Such compositions preferably include, for instance, solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others.

Another example of a pharmaceutically acceptable non-toxic composition in accordance with the present invention, includes an injectable formulation. An injectable formulation can be prepared in conventional forms, for instance, either as liquid solutions or suspensions, solid forms suitable for solution or suspension in liquid prior to injection, or as an emulsion. Suitable excipients include, for example, water, saline, dextrose, glycerol, mannitol, 1,3-butanediol, Ringer's solution, an isotonic sodium chloride solution or ethanol. According to another example, an injectable suspension can be prepared using one or more appropriate liquid carriers, suspending agents and the like. Certain pharmaceutical compositions for injection can be presented in unit dosage form, e.g., in ampules or in multi dose containers. Certain pharmaceutical compositions for injection include, for example, suspensions, solutions or emulsions in oily or aqueous vehicles, and may contain one are more pharmaceutically acceptable excipient agents such as, for instance, one or more suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

In addition to administration of a progestogen hormone, the formulations (e.g., inhalation formulations) and methods of the present invention may further comprise administration of one or more additional therapeutic agents aimed at the treatment of glucocorticoid insensitivity related diseases or disorders, or conditions, as discussed herein. Examples of additional therapeutic agents include, for example, glucocorticoid (e.g., hydrocortisone, cortisone acetate, dexamethasone, prednisone, prednisolone, methylprednisolone, betamethasone, triamcinolone, beclometasone, Paramethasone, fluticasone, fludrocortisone acetate, deoxycorticosterone acetate, Fluprednisolone, fluticasone propionate, budesonide, beclomethasone dipropionate, flunisolide and triamcinolone acetonide, an androgen (e.g., dehydroepiandrosterone (DHEA)), an estrogen (e.g., estradiol), immunosuppressive or immunomodulators agents (e.g., cyclosporine, methotrexate, gold, 6-mercaptopurine, biologic products such as infliximab, etanercept, and adalimumab, intravenous immunoglobulin and Mepolizumab), and calcineurin inhibitors (e.g., cyclosporin, tacrolimus), p38 MAP kinase inhibitors, JNK inhibitors (decrease AP1), Vitamin D, MIF inhibitors, Histone deacetylate-2 activators, Theophylline, Phosphoinositide-3-kinase-δ inhibitors, leukotriene modifiers, long-acting beta agonists, antioxidants, iNOS inhibitors, muscarinic receptor antagonist, bronchodilators, anticholinergic agents, narrow spectrum kinase inhibitors, and P-glycoprotein inhibitors, and combinations thereof.

The other therapeutic agents, when employed in combination with the agents described herein, can be used, for example, in those amounts indicated in the Physicians' Desk Reference (PDR) or as otherwise determined by one of ordinary skill in the art. The amount of an agent used with non-oral routes is preferably determined based upon corresponding serum concentration level of an oral dosage or containing a quantity of the active compound in an amount sufficient to alleviate the symptoms of the treated subject. In the formulations and methods provided herein, such other pharmaceutical agent(s) can be administered prior to, simultaneously with, or following the administration of the compounds provided herein.

Therapeutic effects of the use of a glucocorticoid sensitizer include any, but not limited to, dosing-sparing of concurrent treatment drugs above, better responsive or tolerant to concurrent treatment drugs, achieving efficacy by using lower dose of concurrent treatment drugs, preventing individuals at risk for developing refractory responses or resistance of concurrent treatment drugs, achieving optimal immune-functions, easier responses after tapering or withdrawal of concurrent treatment drugs, or prolonged administration of concurrent treatment drugs, decreased risks for developing drug-related adverse events due to concurrent treatment drugs, and combinations thereof.

Examples 1 and 2 demonstrate the establishment of study models in evaluating steroid sensitivity and steroid resistance, i.e., PHA-induced IL-2 production and IL-2/4 induced steroid resistance in human peripheral blood mononuclear cells (PBMCs) from healthy male smokers.

Examples 3, 4 and 5 demonstrate that progestogen reverses corticosteroid resistance and improves corticosteroid sensitivity under a steroid-resistant condition (i.e., IL-2/4 induced).

Examples 6, 7 and 8 demonstrate that progestogen improves corticosteroid sensitivity under a non-steroid-resistant condition (i.e., PHA-induced IL-2 production without adding IL-2/4).

It has also been surprisingly discovered, in accordance with the present invention, that the effects of the use of a glucocorticoid sensitizer include, but are not limited to, steroid-sparing in corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune-functions, easier responses for a patient when steroid administration is tapered or withdrawn, or prolonged administration of corticosteroids, decreased risks for developing corticosteroid-related adverse events such as opportunistic infections, bone loss, pathologic fracture, diabetes, cataract, and combinations thereof.

### In Vitro Screening Materials and Methods

### Overview

IL-2/4 induced steroid resistance in human peripheral blood mononuclear cells (PBMCs) is a well-recognized study model to evaluate potential modifiers of steroid resistance and sensitivity (Kam, J. C. etc. Combination IL-2 and IL-4 reduces glucocorticoid receptor-binding affinity and T cell response to glucocorticoids. J. Immunol. 1993. 151: 3460-3466. Irusen, E. etc. p38 mitogen-activated protein kinase-induced glucocorticoid receptor phosphorylation reduces its activity: role in steroid-insensitive asthma. J. Allergy Clin. Immunol 2002. 109: 649-657. Creed T J etc. The Effects of Cytokines on Suppression of Lymphocyte Proliferation by Dexamethasone. J Immunol 2009; 183; 164-171). The dexamethasone inhibition of lymphocyte proliferation and cytokine assay correlates well with the outcome of steroid therapy in patients with inflammatory diseases. The suppression of cytokine releases by dexamethasone (also referred to herein in this application as “Dexamethasone”) in healthy volunteers is used to measure changes in steroid sensitivity and steroid resistance. Following PBMCs stimulation with mitogen phytohaemagglutinin (PHA), there is a strong correlation between cytokine secretion levels and steroid resistance as well as steroid sensitivity in vitro. Addition of IL-2, IL-4 and TNF-reduce steroid sensitivity.

The objective of one study was to evaluate the compounds' effect on reversing corticosteroid resistance, measured by an increase in the ability of dexamethasone to inhibit PHA-induced IL-2 release in the IL-2 and IL-4 induced steroid resistant model.

The compounds' effects were assessed in improving corticosteroid sensitivity, measured by IC50 improvement, steroid-sparing, achieving a similar anti-inflammatory efficacy by using a lower dose of corticosteroid, better responsiveness and combination of synergetic effects in the PHA-induced IL-2 release in PBMCs.

### Materials

PBMCs (peripheral blood mononuclear cells; PBMCs) separation system: Accuspin system-Histopaque from GEHealthcare Bio-Sciences AB (US); RPMI-1640 Medium from HyClone, Beijing China, dimethyl sulfoxide (DMSO) from Sigma (US), 17α-HYDROXYPROGESTERONE CAPROATE (17HPC) (CAS No: 630-56-8), MEDROXYPROGESTERONE ACETATE (MPA) (CAS No. 71-58-9), natural Progesterone (P4) (CAS No. 57-83-0), Dexamethasone (Dexamethasone) (CAS No: 50-02-2) and PHA from Sigma Ltd (US); recombinant IL-2 and IL-4 from PeproTech, IL-2 immunosorbent assay kit (ELISA for IL-2) from ExCell Biology, Shanghai China; anti-human anti-CD3 and anti-CD28 from R&D Systems (US).

### Isolation of PBMCs

PBMCs were isolated from human blood buffy coats provided by a regional blood center. Random buffy coat cells from male donors were a by-product of blood processed for clinical use and no details (i.e., personal identification and background) were provided except for tobacco use. Almost all of the male blood donors were cigarette smokers. PBMCs were separated using a porous high density polyethylene barrier (HISTOPAQUE from GEHealthcare Bio-Sciences AB (US). After centrifugation of blood samples in each tube (35 minutes at 800×g at room temperature, or RT), PBMCs were collected and washed twice with Hank's buffered saline solution (HBSS). PBMCs were resuspended in RPMI-1640 medium containing 10% fetal calf serum (FCS) and 15 mM glutamine and cells were counted and plated.

### Culture of Cells

PBMCs (2×10⁶) were incubated with or without IL-2 (13 ng/ml) and IL-4 (6.5 ng/ml) for 48 hours in RPMI-1640 medium containing 10% FCS and 2 mM glutamine. PBMCs were counted and plated again at 10⁷ cells/ml before stimulating with or without 17α-HYDROXYPROGESTERONE CAPROATE (17HPC), Progesterone (P4) and MEDROXYPROGESTERONE ACETATE (MPA) for 12 hours prior treatment of Dexamethasone dexamethasone. IL-2 and IL-4 stimulated PBMCs were also plated at a concentration of 10⁶ cells/ml in 96-well plates ready for PHA (15 μg/mL, 24 hours) stimulation of cytokine release and detection by ELISA.

### Sandwich-ELISA (Enzyme Linked Immunosorbent Assay)

PBMC Cells (10⁶ cells/ml) were plated in 96-well plates and stimulated with or without dexamethasone (10⁻¹² M to 10⁻⁵ M) for 1 hour before transferring the cells in a 96-well plate with or without PHA (15 μg/mL) 24 hours at 37° C., 5% CO₂. Serial dilutions of standards and PBMCs' supernatants were measured by enzyme linked immunosorbent assay to determine IL-2 at baseline and its levels after 17HPC, P4 and MAP treatments. Optical density was measured at 450 nm and corrected with 550 nm. The concentration of TL-2 was calculated using the standard curve and taking into account the supernatant dilution used. Detection limit for IL-2 is 4.0 pg/ml.

### Statistical Analysis

Data are expressed as mean±SD. The efficacies of the drug treatments were analyzed by paired t-test. One-way analysis of variance was used to compare three or more matched groups and 95% CI was performed to present groups differences. All graphs indicate mean values of results or % inhibition of PHA-stimulated IL-2. IC50 values were calculated by using a sigmoidal model (BioDataFit). A p value <0.05 was considered statistically significant.

### Results

### Simultaneous Measurement of Steroid Sensitivity in PBMCs (10⁶ Cells/ml)

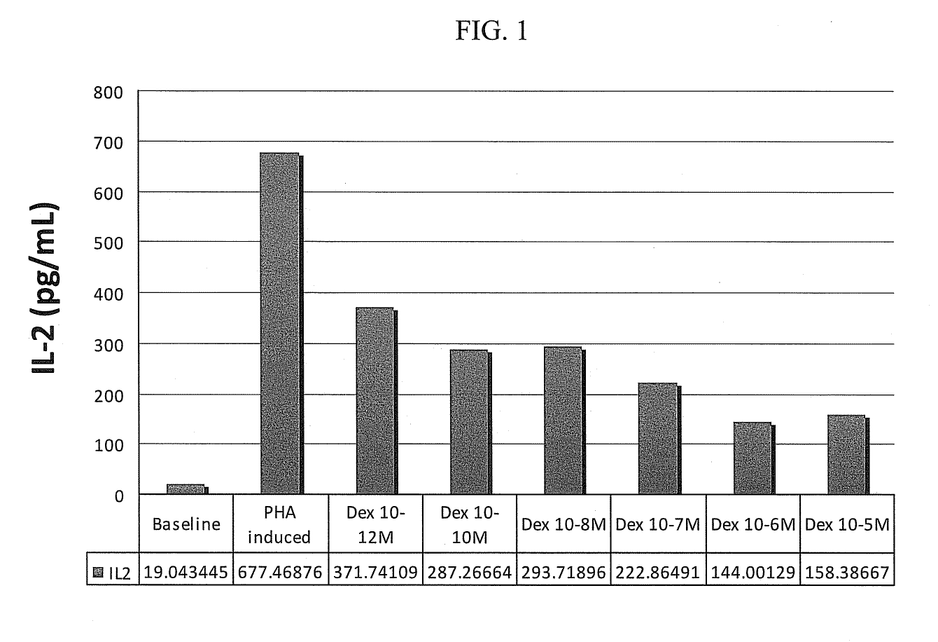

To measure glucocorticoid sensitivity simultaneously, PBMCs (10⁶ cells/ml) were plated in 96-well plates and stimulated with or without serial dilutions of dexamethasone (10⁻¹² M to 10⁻⁵ M) for 1 hour, and then were subsequently with PHA (15 μg/mL) for 24 hours at 37° C., 5% CO₂. IL-2 levels were quantified using ELISA. Results in FIG. 1 include IL-2 levels at baseline, after PHA stimulation and dose-dependent inhibition of IL-2 by dexamethasone. The levels of IL-2 in PBMCs were 19±25 pg/ml at baseline in healthy Male smokers (n=11), 677±447 pg/ml after PHA stimulation PHA (n=20), 371±447 pg/ml at dexamethasone 10⁻¹² M (n=11), 287±313 pg/ml at dexamethasone 10⁻¹⁰ M (n=14), 293±338 pg/ml at dex 10⁻⁸M (n=17) and 144±157 pg/ml at dexamethasone 10⁻⁶M (n=17). The Dexamethasone and 17HPC treatments have no significant effect in basal IL-2 level (data not shown). FIG. 1 thus shows IL-2 levels (mean) at baseline, after PHA stimulation and effect of dexamethasone on PHA-induced IL-2 production (n=20) (p<0.001). Dexamethasone shows a significant, dose-response inhibition of IL-2 production (FIG. 1).

### Example 1

### Addition of IL-2 and IL-4 Reduces Steroid Sensitivity or Induces Steroid Resistance Among Male Smokers

IL-2/4 induced steroid resistance in PBMCs, a well-recognized study model, was used to evaluate potential modifiers of steroid resistance and sensitivity. PBMCs from healthy smokers were collected. Corticosteroid insensitivity or resistance was induced by adding IL-2 and IL-4 in peripheral blood mononuclear cells (PBMCs) from healthy male smokers (n=˜11). PBMCs (10⁶ cells/ml) stimulated with or without IL-2 (13 ng/ml) and IL-4 (6.5 ng/ml) were cultured in 96-well plates for 48 hours and subsequently being exposed serial dilutions of dexamethasone (10⁻¹⁰ M, 10⁻⁸ M to 10⁻⁶ M) for 1 hour, and then were stimulated with PHA (15 μg/mL) for 24 hours at 37° C., 5% CO2. IL-2 levels were quantified using ELISA. Percentage of inhibition on PHA-induced IL-2 production was calculated as % Inhibition=1−(IL-2 with Dexamethasone/IL-2 without Dexamethasone).

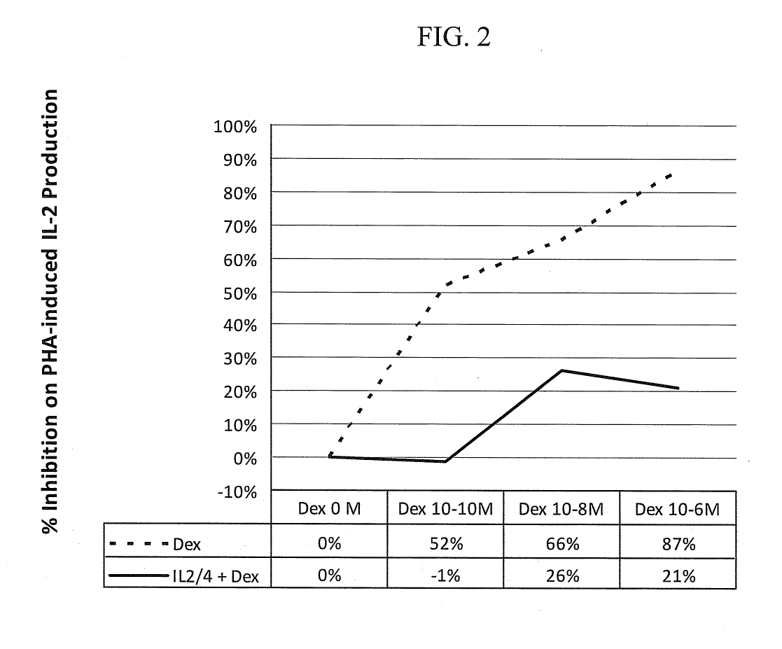

The results depicted in FIG. 2 show that inhibition ability of dexamethasone on PHA-induced IL-2 production was significantly reduced with addition of IL-2 and IL-4 among male smokers. For example, % inhibition with low dose Dexamethasone 10⁻¹⁰ M was completely lost: 52% vs. no inhibition, and with higher dose Dexamethasone 10⁻⁶ M: 87% vs. 21%, a significant reduction. Addition of IL-2 and IL-4 reduces steroid sensitivity or induces steroid resistance among male smokers, a valid steroid-resistant model/condition.

### Example 2

### Progestogen Improves Corticosteroid Sensitivity or Reverses Corticosteroid Resistance Among Male Smokers

Corticosteroid insensitivity or resistance can be reversed pharmacologically. We investigated the effects of progestogen drug class which is currently unknown for its function in reversing steroid resistance, and test Progestogen drugs of 17α-HYDROXYPROGESTERONE CAPROATE (17HPC), MEDROXYPROGESTERONE ACETATE (MPA) and natural Progesterone (P4) on their effects in improving glucocorticoid sensitivity in peripheral blood mononuclear cells (PBMCs) from healthy male smokers.

PBMCs (10⁶ cells/ml) stimulated with IL-2 (13 ng/ml) and IL-4 (6.5 ng/ml) were cultured in 96-well plates for 48 hours and subsequently stimulated with 17HPC (10⁻¹⁰ M, M and 10⁻⁵ M) or P4 or MPA ((10⁻¹⁰ M, 10⁻⁸ M and 10⁻⁵ M) for 12 hours before being exposed with or without low and high doses of dexamethasone (10⁻¹⁰ M and 10⁻⁶ M) for 1 hour, and then were subsequently with PHA (15 μg/mL) for 24 hours at 37° C., 5% CO2 (n=11 for the combinations of 17HPC+Dexamethasone groups). IL-2 levels were quantified using ELISA. A 10% increase in maximal Dexamethasone inhibition (Imax) under a steroid-resistant condition represents a clinical meaningful improvement (Creed T J etc. The Effects of Cytokines on Suppression of Lymphocyte Proliferation by Dexamethasone. J Immunol 2009; 183; 164-171).

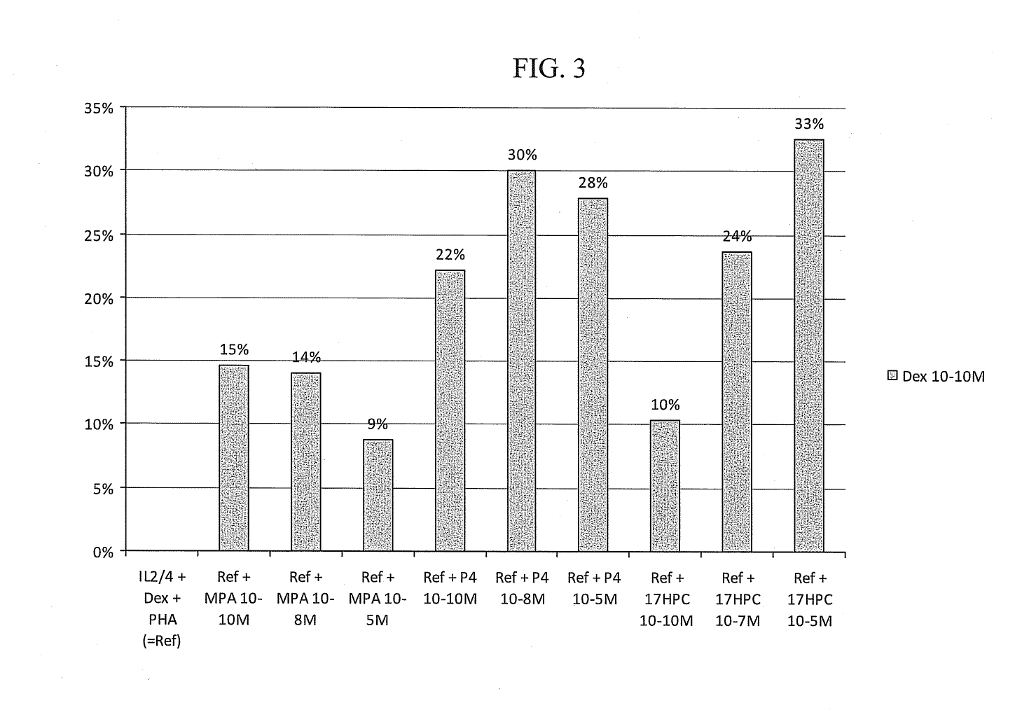

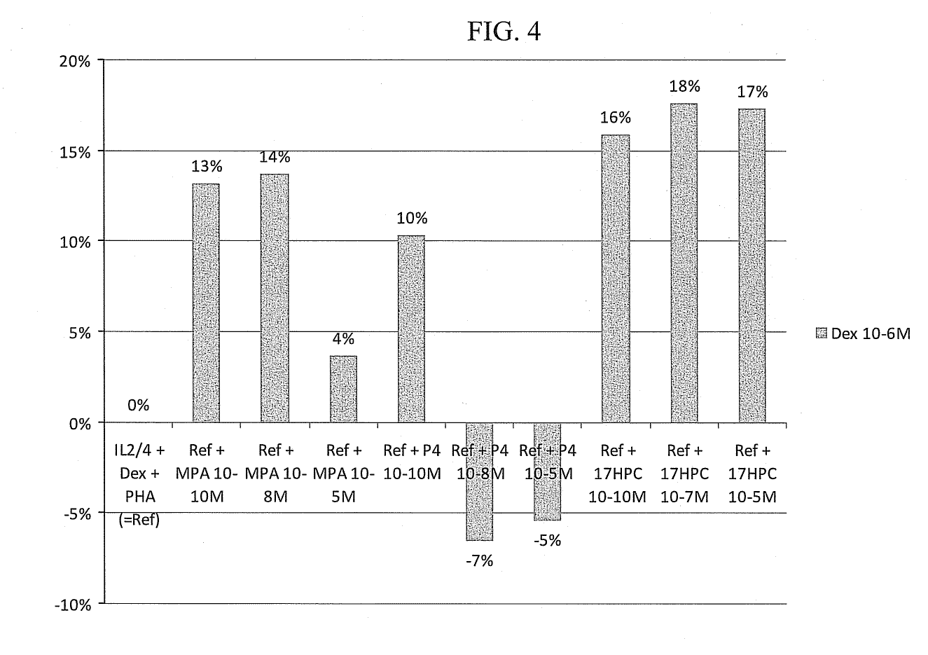

The results depicted in FIGS. 3-4 show that progestogen consistently improves corticosteroid insensitivity by all three progestogen agents (17HPC, P4 and MPA) when the low dose of dexamethasone is used. The Imax improves from 9% to 33%. FIG. 3 depicts progestogen's effects (% Imax) in reversing steroid resistance: comparing 17HPC, P4 and MPA under low dose Dexamethasone (10⁻¹⁰ M). FIG. 4 depicts progestogen's effects (% Imax) in reversing steroid resistance: comparing 17HPC, P4 and MPA under high dose dexamethasone (10⁻⁶ M).

The effects in reversing corticosteroid resistance are observed when the high dose of Dexamethasone is used (FIG. 4). Each progestogen agent has its own dose-response pattern. Among the three tested drugs, 17HPC has the best treatment effects, i.e., highest improvement rate (of 18%) and consistency at all dose levels (16-18%).

Progestogen (e.g., 17HPC, P4 and MPA) thus has the surprising and unexpected effect of reversing glucocorticoid resistance and improving glucocorticoid sensitivity in cigarette smokers. Progestogen, therefore, can be used to treated smoking-induced glucocorticoid resistance diseases such as, for instance, chronic obstructive pulmonary disease (COPD).

### Example 3

### 17HPC Reverses Corticosteroid Resistance Among Male Smokers

PBMCs (10⁶ cells/ml) stimulated with IL-2 (13 ng/ml) and IL-4 (6.5 ng/ml) were cultured in 96-well plates for 48 hours and subsequently stimulated with 17HPC (10⁻¹⁰ M, 10⁻⁷ M and 10⁻⁵ M) for 12 hours before being exposed with or without three doses of dexamethasone (10⁻¹⁰ M, 10⁻¹⁸M and 10⁻⁶ M) for 1 hour, and then were subsequently with PHA (15 μg/mL) for 24 hours at 37° C., 5% CO2 (n=11). IL-2 levels were quantified using ELISA.

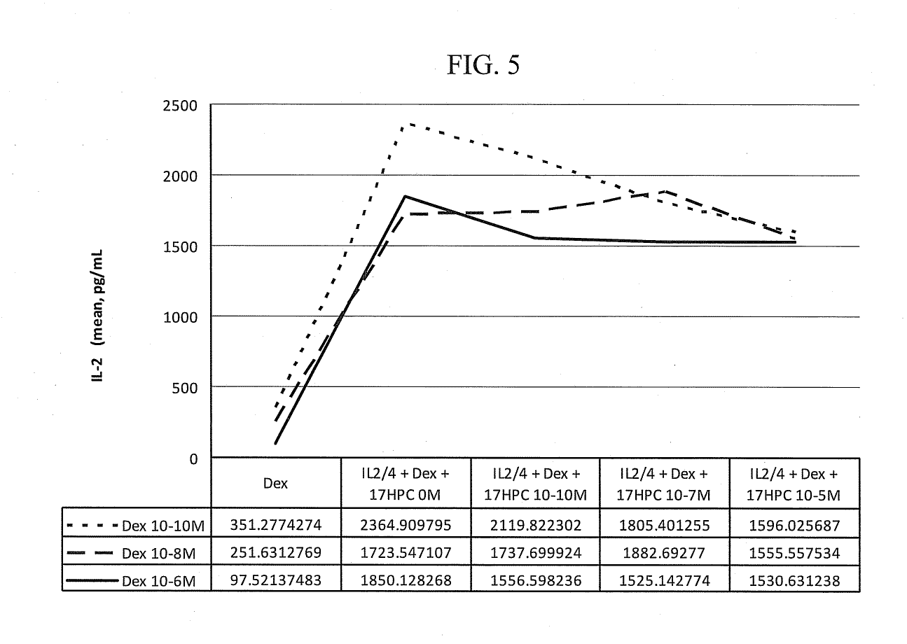

FIG. 5 shows that the addition of IL-2 and IL-4 reduced steroid sensitivity significantly at all three Dexamethasone concentrations. The improvement of dexamethasone inhibition of PHA-induced IL-2 release is achieved by adding 17HPC. 17HPC reverses the glucocorticoid insensitivity in a dose-response pattern. 17HPC thus restores corticosteroid sensitivity. For example, PHA-induced IL-2 level with Dexamethasone 10⁻¹⁰ M, but without 17HPC was 2364 pg/mL vs. significantly improved cytokine suppression of 2119, 1805 and 1595 pg/mL after adding 17HPC at 10⁻¹⁰ M, 10⁻⁷M and 10⁻⁵M respectively (p<0.05 in both 17HPC 10⁻⁷ M and 10⁻⁵M groups).

FIG. 6 shows individual responses before-and-after 17HPC treatment when the high dose of Dexamethasone 10⁻⁶ M was given. Ten out of 11 subjects had a more than 10% improvement (in % maximal Dexamethasone inhibition) after 17HPC treatment, and only one subject (#6) had no improvement (p<0.05 in Chi Square Test for all three 17HPC dose groups). 17HPC thus reverses steroid resistance and individual response patterns.

### Example 4

### Individual Response Patterns with Three Progestogen Agents: 17HPC, P4 and MPA

Example 3 above showed that Progestogen reverses corticosteroid resistance among male smokers. FIG. 7-9 compares individual response patterns with three progestogen agents: 17HPC, P4 and MPA with the same Dexamethasone dose 10⁻¹⁰ M:

The results depicted in FIG. 7 show that 9 out 11 subjects had a more than 10% improvement in maximal Dexamethasone inhibition after receiving a dose of 17HPC, which is consistent with the results presented in FIG. 6.

The results depicted in FIG. 8 show that 6 out of 8 subjects had a more than 10% improvement in maximal Dexamethasone inhibition after receiving a dose of natural progesterone, which is similar to 17HPC.

The results depicted in FIG. 9 shows that MPA treatment leads a total different response pattern: a “split” response. A sub-group had a great improvement up to 58% while another sub-group presented with a worsening in corticosteroid sensitivity, a reduction up to 88%.

### Example 5

### Progestogen (e.g., 17HPC) Improves Corticosteroid Sensitivity Under a Non-Steroid Resistant Condition

To determine the effect of add-on treatment of 17HPC on glucocorticoid sensitivity simultaneously, PBMCs (10⁶ cells/ml) were plated in 96-well plates and stimulated with 17HPC (10⁻¹¹ M to 10⁻⁵ M) for 12 hours before being exposed with or without serial dilutions of dexamethasone (10⁻¹² M to 10⁻⁶ M) for 1 hour, and then were subsequently with PHA (15 μg/mL) for 24 hours at 37° C., 5% CO2. IL-2 levels were quantified using ELISA. IC₅₀ values were calculated by using a sigmoidal model (BioDataFit). The value (of IL-2=734) from negative control (i.e., cell+PHA without Dexamethasone or 17HPC) was artificially set as Dexamethasone-¹⁸M or 17HPC⁻¹⁷ M (i.e., assuming drug concentration≈0 M) to fit the sigmoidal model when calculating Dexamethasone-IC₅₀ and 17HPC-IC₅₀ (N=14).

The effects of 17HPC on dexamethasone sensitivity measured by IL-2 inhibition in smokers are shown in Table 1 and FIG. 10. FIG. 10 shows add-on effect of 17HPC is improvement of steroid sensitivity. 17HPC, especially at lower concentrations, significantly enhances steroid sensitivity measured by Dexamethasone-IC₅₀, which is improved from IC₅₀=7.5 (Dexamethasone only) to 10.2-12.0 (Dexamethasone+17HPC) (p=0.0052 in ANOVA). The higher dose of 17HPC (at 17HPC 10⁻⁵ M) had minimal or no effect.

TABLE 1
17HPC effect in improving corticosteroid sensitivity (mean IL-2 pg/mL and IC₅₀)

| | Dexamethasone | 17HPC | 17HPC | 17HPC | 17HPC | PR2005 |
|---|---|---|---|---|---|---|
| Dexamethasone | only | 10⁻¹¹M | 10⁻¹⁰M | 10⁻⁷M | 10⁻⁵M | IC50 |
| 0 | 734.6692754 | 448.2208447 | 420.6095024 | 466.3103053 | 225.5846837 | 6.434401 |
| 10⁻¹²M | 371.7410856 | 275.1887924 | 132.298398 | 111.5503515 | 176.2274866 | 10.92452 |
| 10⁻¹⁰M | 312.6599339 | 164.3874069 | 177.7437283 | 104.5164134 | 138.578293 | 11.64492 |
| 10⁻⁸M | 398.132707 | 167.8640405 | 173.5466553 | 229.3011166 | 136.1965467 | 6.242864 |
| 10⁻⁶M | 160.6293856 | 9.050299835 | 27.18227874 | 9.914185277 | 24.6061311 | N/A |
| Dexamethasone | 7.509749 | 12.01514 | 10.20306 | N/A | 7.595664 | |
| IC50 | | | | | | |

### Example 6

### Progestogen (e.g., 17HPC) has Significant Steroid-Sparing Effects

To determine effect of add-on treatment of 17HPC on glucocorticoid sparing simultaneously, PBMCs (10⁶ cells/ml) were plated in 96-well plates and stimulated with 17HPC (10⁻¹¹ M to 10⁻⁵ M) for 12 hours before being exposed with or without serial dilutions of dexamethasone (10⁻¹²M to 10⁻⁶ M) for 1 hour, and then were subsequently with PHA (15 μg/mL) for 24 hours at 37° C., 5% CO2. IL-2 levels were quantified using ELISA. The % inhibition of PHA stimulation of IL-2 (pg/mL) by 17HPC and/or Dexamethasone (Dexamethasone) in PBMCs was calculated. The value (of IL-2=734.7) from the negative control (i.e., cell+PHA without any drug treatment) was set as zero % inhibition, and all other % inhibition values were derived from the formula: (1−(treatment IL-2 level/714.5)×100%, (N=14) (note: 714.5 ng/mL (PHA-induced IL-2) was the mean value from the 14 subjects).

The add-on of 17HPC can achieve similar efficacy by using a lower dose of corticosteroid (Table 2). The percentage of suppression of IL-2 releases by higher dose of 10⁻⁶ M dexamethasone (Imax) in healthy smokers is 78%. The ‘add-on’ treatment of 17HPC will significantly reduce the dose requirement for Dexamethasone. Table 2 shows that add-on of the low doses of 17HPC (10⁻¹¹ M or 10⁻¹⁰ M) will achieve a similar anti-inflammatory effect (≧78% IL-2 inhibition) when comparing to Dexamethasone 10⁻⁶ M), i.e., only using the 1/1,000^{th} to 1/100,000th of the original Dexamethasone dose, a substantial steroid-sparing effect. Therefore, the add-on of 17HPC may prevent individuals at risk for developing refractory responses or resistance or exacerbations or tolerance to corticosteroids as well as improving safety profiles.

TABLE 2
Add-on Effect of 17HPC: Steroid-sparing effects measured
by % inhibition of PHA stimulation of IL-2

| | Dexamethasone | 17HPC | 17HPC | 17HPC | 17HPC |
|---|---|---|---|---|---|
| | only | 10⁻¹¹ M | 10⁻¹⁰ M | 10⁻⁷ M | 10⁻⁵ M |
| Dexamethasone | 0% | 39% | 43% | 37% | 69% |
| 0 M | | | | | |
| Dexamethasone | 49% | 63% | 82% | 85% | 76% |
| 10⁻¹² M | | | | | |
| Dexamethasone | 57% | 78% | 76% | 86% | 81% |
| 10⁻¹⁰ M | | | | | |
| Dexamethasone | 46% | 77% | 76% | 69% | 81% |
| 10⁻⁸ M | | | | | |
| Dexamethasone | 78% | 99% | 96% | 99% | 97% |
| 10⁻⁶ M | | | | | |

Values represent the % inhibition of PHA stimulation of IL-2 (pg/mL) by 17HPC only, Dexamethasone only and 17HPC plus Dexamethasone at different concentrations in the PBMC model.

### Example 7

### Add-on of Progestogen (e.g., 17HPC) Leads to a Better Treatment Responsiveness, and the Combination of 17HPC with Dexamethasone Results in Synergetic Effects

To determine the effect of add-on treatment of 17HPC on glucocorticoid sparing simultaneously, PBMCs (10⁶ cells/ml) were plated in 96-well plates and stimulated with 17HPC (10⁻¹¹ M to 10⁻⁵ M) for 12 hours before being exposed with or without serial dilutions of dexamethasone (10⁻¹²M to 10⁻⁶ M) for 1 hour, and then were subsequently with PHA (15 μg/mL) for 24 hours at 37° C., 5% CO2. IL-2 levels were quantified using ELISA. The % inhibition of PHA stimulation of IL-2 (pg/mL) by 17HPC and/or Dexamethasone (Dexamethasone) in PBMCs was calculated. The value (of IL-2=734.7) from the negative control (i.e., cell+PHA without any drug treatment) was set as zero % inhibition, and all other % inhibition values were derived from the formula: (1−(treatment IL-2 level/714.5)×100%, (N=14).

The maximal anti-inflammatory effect of Dexamethasone is 78% inhibition of PHA-induced IL-2 production at 10⁻⁶ M. The ‘add-on’ treatment of 17HPC produces a significantly better responsiveness and results in near 100% suppression of PHA induced IL-2 (FIG. 11). FIG. 11 thus depicts a better treatment responsiveness with the 17HPC add-on.

Further, the combination of 17HPC with Dexamethasone consistently increases Dexamethasone's anti-inflammatory effects, and is better than their uses individually. FIG. 12 shows that the combination leads to a synergetic effect, with 25-37% improvements in Dexamethasone efficacy. FIG. 12 thus depicts synergetic effects of combination of 17HPC with Dexamethasone.

### Example 8

### Add-on of Other Progestogen Compounds (e.g., P4 and MPA) Shows Similar Effects in Enhancing Glucocorticoid Sensitivity

To determine effect of add-on treatment of MEDROXYPROGESTERONE ACETATE (MPA) and natural Progesterone (P4) on glucocorticoid sparing simultaneously, PBMCs (10⁶ cells/ml) were plated in 96-well plates and stimulated with P4 or MPA (10⁻¹⁰ M, 10⁻⁸ M and 10⁻⁵ M) for 12 hours before being exposed with or without dexamethasone (10⁻¹¹ M to 10⁻⁸ M) for 1 hour, and then were subsequently with PHA (15 μg/mL) for 24 hours at 37° C., 5% CO2 (n=6 for the combinations of P4 or MAP+Dexamethasone). IL-2 levels were quantified using ELISA. The % inhibition of PHA stimulation of IL-2 (pg/mL) by P4, or MPA, and/or Dexamethasone (Dexamethasone) in PBMCs was calculated. The value (of IL-2=765) from the negative control (i.e., cell+PHA without any drug treatment, n=25) was set as zero % inhibition, and all other % inhibition values were derived from the formula: (1−(treatment IL-2 level/765)×100%.

Table 3 and Table 4 show that both P4 and MPA have similar effects in enhancing glucocorticoid sensitivity such as steroid-sparing and synergetic effects of combination. For examples, the percentage of suppression of IL-2 releases by 10⁻⁸ M dexamethasone is improved from 67% to 96% when MPA 10⁻⁸M is added (P=0.035 in pared T-Test). The add-on of the low dose of either P4 or MPA (10⁻¹⁰ M) will achieve a similar anti-inflammatory effect (≧86% IL-2 inhibition) when comparing to Dexamethasone 10⁻⁶ M), i.e., only using the 1/100^{th} of the original Dexamethasone dose, a substantial steroid-sparing effect.

TABLE 3
Add-on effects of Medroxyprogesterone Acetate (MPA) on enhancing
glucocorticoid
sensitivity (Mean IL-2 pg/mL and % inhibition of PHA-induced
IL-2 production

| | | MPA | MPA | MPA |
|---|---|---|---|---|
| | | 10⁻¹⁰M | 10⁻⁸M | 10⁻⁵M |
| PHA | 765.0 | 206.1 | 166.9 | 39.8 |
| | (0.0%) | (73%) | (78%) | (95%) |
| DEXAMETHASONE | 216.1 | | 129.1 | |
| 10E−11M | (72%) | | (83%) | |
| DEXAMETHASONE | 251.6 | 44.7 | 29.2 | 12.3 |
| 10E−8M | (67%) | (94%) | (96%) | (98%) |
| DEXAMETHASONE | 109.6 | | | |
| 10E−6M | (86%) | | | |

TABLE 4
Add-on effects of natural progesterone (P4) on enhancing
glucocorticoid sensitivity (Mean IL-2 pg/mL and % inhibition of
PHA-induced IL-2 production)

| | | P4 | P4 | P4 |
|---|---|---|---|---|
| | | 10⁻¹⁰M | 10⁻⁸M | 10⁻⁵M |
| PHA | 765.0 | 506.9 | 486.2 | 113.8 |
| | (0.0%) | (34%) | (36%) | (85%) |
| DEXAMETHASONE | 216.1 | | 100.8 | |
| 10E−11M | (72%) | | (87%) | |
| DEXAMETHASONE | 251.6 | 59.6 | 91.4 | 81.2 |
| 10E−8M | (67%) | (92%) | (88%) | (89%) |
| DEXAMETHASONE | 109.6 | | | |
| 10E−6M | (86%) | | | |

### Example 9

Exemplary particle size reduction experiments were performed, and characterization of API particle size distribution was determined. Experiments were performed, comparing the particle size of bulk material, compared to the particle size after 8 to 25 cycles of milling with the surfactant Tween 80 (15%). FIG. 13 depicts exemplary results of characterization of the bulk material 17-HPC (showing percent change in weight of the bulk material relative to percent changes in relative humidity, or RH %) using Dynamic Vapor Sorption (DVS). FIG. 14 depicts exemplary results of characterization of the bulk material 17-HPC using X-ray powder diffraction (XRPD) characterization.

Referring to Table 5, experiments were performed with a suspension comprising the active pharmaceutical ingredient (the API), i.e., 17-HPC was the API in these experiments, wherein 17-HPC was present at approximately five percent (5%) w/w in water; and the suspension also comprised the surfactant Tween 80 at approximately 15% in these experiments (processing conditions conducted at Pressure=90 bar). As shown in Table 5, particle size reduction of 17-HPC was observed by using a surfactant (in this case, Tween 80 at fifteen percent). API particle size consistently decreased after an increasing number of cycles, using a suspension of bulk starting material with the surfactant Tween 80 present in the suspension. Referring again to Table 5, these exemplary particle size reduction experiments demonstrate that API particle size consistently decreases after an increasing number of cycles. Particle size measurements are expressed in terms of Dv(10), Dv(50), and Dv(90), wherein Dv(10) refers to the particle size below which 10% of the volume of material exists; Dv(50) refers to the particle size below which 50% of the volume of material exists; and Dv(90) refers to the particle size below which 90% of the volume of material exists).

Referring to FIG. 15, an exemplary 17-HPC particle size distribution profile is shown after 8 to 25 cycles of milling of a suspension containing the 17-HPC with the surfactant Tween 80 (15%), compared to the particle size distribution profile for the bulk material.

TABLE 5
Particle size reduction of 17-HPC by using a surfactant
(in this case, Tween 80 at fifteen percent). API particle size
consistently decreased after an increasing number of cycles,
using a suspension of bulk starting material with the surfactant
Tween 80 present in the suspension. Note for the processing
conditions: 5% (w/w) - 17-HPC in water; 15% Tween 80;
Pressure = 90 bar.
API particle size

| | | Bulk | After | After | After |
|---|---|---|---|---|---|
| | | material | 8 cycles | 16 cycles | 25 cycles |
| Particle Size | Dv(10) | 6.345 μm | 1.015 μm | 1.071 μm | 1.014 μm |
| | Dv(50) | 56.762 μm | 3.021 μm | 2.574 μm | 2.255 μm |
| | Dv(90) | 307.607 μm | 7.302 μm | 5.347 μm | 4.517 μm |
| | Span | 5.307 | 2.081 | 1.662 | 1.553 |

### Example 10

Characterization of API particle size distribution was performed using a suspension prepared without a surfactant (i.e., no surfactant was included). Referring to Table 6, experiments were performed with a suspension comprising the active pharmaceutical ingredient (the API), i.e., 17-HPC in these experiments, in water. The suspension did not comprise any surfactant, i.e., without Tween 80. With this suspension, the API product (17-HPC) was forced to come into contact with water. Referring again to Table 6, particle size measurements are expressed in terms of Dv(10), Dv(50), and Dv(90), wherein Dv(10) refers to the particle size below which 10% of the volume of material exists; Dv(50) refers to the particle size below which 50% of the volume of material exists; and Dv(90) refers to the particle size below which 90% of the volume of material exists).

TABLE 6
Characterization of API particle size distribution using
a suspension prepared without a surfactant.

| | | | PGC2011- | | |
|---|---|---|---|---|---|
| | | PGC2011- | 020PD | PGC2011- | PGC2011- |
| | | 018PD | After | 021PD | 022PD |
| | | After | 25 cycles | After | After |
| | | 10 cycles | 2.5% | 40 cycles | 55 cycles |
| | | 2.5% (w/w), | (w/w), | 2.5% (w/w), | 2.5% (w/w), |
| | | P = 90 bar | P = 90 bar | P = 90 bar | P = 120 bar |
| Particle | Dv (10) | Not analyzed | 1.078 μm | 1.070 μm | 1.197 μm* |
| Size | Dv(50) | Not analyzed | 2.780 μm | 2.521 μm | 3.720 μm* |
| | Dv(90) | Not analyzed | 5.626 μm | 4.862 μm | 8.322 μm* |
| | Span | Not analyzed | 1.636 | 1.505 | 1.915* |

*Higher than expected due to agglomeration (the samples were slightly wet)—confirmed by particle size results of the powder obtained after SD

### Example 11

Exemplary particle size reduction experiments, and characterization of API (i.e., 17-HPC) particle size distribution, using a suspension prepared without a surfactant. Referring to Table 7, particle size reduction of 17-HPC was observed without using any surfactant. Experiments were performed with a suspension comprising the active pharmaceutical ingredient (the API), i.e., 17-HPC in these experiments, in water and without a surfactant. FIG. 16 depicts an exemplary API particle size distribution profile (17-HPC is the active ingredient) after a spray-drying (SD) process. Referring again to FIG. 16, and also to Table 7, an exemplary 17-HPC particle size distribution profile was obtained after approximately 10 to 55 cycles of milling. API particle size was measured after spray drying (SD). Particle size measurements are expressed in terms of Dv(10), Dv(50), and Dv(90), wherein Dv(10) refers to the particle size below which 10% of the volume of material exists; Dv(50) refers to the particle size below which 50% of the volume of material exists; and Dv(90) refers to the particle size below which 90% of the volume of material exists).

### Example 12

After a spray-drying (SD) process was performed, dry powder containing 17-HPC as the active ingredient after particle size reduction were analyzed for impurity profiles. FIG. 17 depicts an exemplary impurity profile of a dry powder obtained after a spray-drying (SD) process, as compared to the impurity profile of the starting bulk material. As shown in FIG. 17, the obtained powders and the starting bulk material have similar impurity profiles, which indicates that the SD process does not generate any extra impurities.

### Example 13

Analysis of 17-HPC dry powder (obtained after particle size reduction and spray drying or SD) was performed. FIG. 18 depicts exemplary results from a dynamic vapor sorption (DVS) analysis of a dry powder obtained after a spray-drying (SD) process. One particular batch (PGC2011-020PD) was selected to be analyzed by DVS. As shown in FIG. 18, based on the DVS analysis (in which percent change in weight was evaluated under conditions of different percent relative humidity, or RH %), it was observed that neither the obtained powders nor the bulk material gained any measurable quantity of water. There was no substantive change in adsorption or desorption even with conditions of increasing percent relative humidity. Referring to FIG. 19, exemplary results are shown of XRPD analysis of a 17-HPC dry powder obtained after particle size reduction and a spray-drying (SD) process. One particular batch (PGC2011-020PD) was selected to be analyzed by XRPD. As shown in FIG. 19, the obtained powder (17-HPC is the active ingredient) and the bulk material have similar XRPD profiles, which indicates that the particle size reduction and spray drying (SD) process does not change the crystalline structure.

### Example 14

Analysis of different exemplary dry powder blend formulations was performed. FIG. 20 depicts the concentration of API per capsule, relative to three different particle sizes, for exemplary dry powder blend formulations obtained after a particle size reduction and spray-drying (SD) process with lactose. It was determined that particularly preferred powder formulations for administration by inhalation comprise the active substance (17-HPC) and a pharmaceutically acceptable excipient (e.g., lactose). Moreover, it was also determined that a particularly preferred composition has the form of a physical mixture, wherein the composition comprises from about five (5) to about fifty (50) weight percent of the excipient; wherein the active substance (17-HPC) has a particle size distribution of from about one nanometer to about ten (10) microns, and wherein the excipient (e.g., lactose) has a particle size distribution of from about fifteen (15) microns to about five-hundred (500) microns.

### Example 15

Measurements were taken to evaluate the relationship between particle size and fine-particle dose (FPD). FIGS. 21 and 22 both depict exemplary results showing a good correlation between particle size and FPD.

TABLE 7
Exemplary results showing characterization of API particle size distribution after spray drying (SD) (powders),
and after 10 to 55 cycles of milling.
API particle size after SD (powders)

| | | | PGC2011-018PD | PGC2011-020PD | PGC2011-021PD | PGC2011-022PD |
|---|---|---|---|---|---|---|
| | | Bulk material | After 10 cycles | After 25 cycles | After 40 cycles | After 55 cycles |
| Particle Size | Dv(10) | 2.398 μm | 1.079 μm | 1.037 μm | 1.017 μm | 1.037μm |
| | Dv(50) | 23.610 μm | 3.560 μm | 2.602 μm | 2.619 μm | 2.434 μm |
| | Dv(90) | 98.799 μm | 8.279 μm | 5.364 μm | 5.658 μm | 4.865 μm |
| | Span | 4.083 | 2.024 | 1.663 | 1.772 | 1.573 |

### Example 16

Referring to Tables 8, 9 and 10, three different exemplary dry powder blends are shown, comprising 17-HPC as the active pharmaceutical ingredient (API), formulated for inhalation delivery. The 17-HPC powder formulations for administration by inhalation comprise the active substance (17-HPC) and a pharmaceutically acceptable excipient (e.g., lactose), which composition has the form of a physical mixture and comprises the excipient, and wherein the active substance (of 17-HPC) has a particle size distribution of from 0.5 to 10 μm for the powder formulations.

TABLE 8
One exemplary powder blend formulation is shown, comprising
17-HPC as the active pharmaceutical ingredient (API).

| | Content in formulation | |
|---|---|---|
| Components | LBC2011-048B | LBC2011-049B |
| SD powder (PGC2011-018PD, | 1% (100 μg*) | 10% (1000 μg*) |
| Dv(50) = 3.56 μm) | | |
| Lactose Monohydrate: Respitose | 89% | 80% |
| ML001 | | |
| Lactose Monohydrate: Lactohale | 10% | 10% |
| LH300 | | |

*Considering a total fill weight of 10 mg per capsule

TABLE 9
Another exemplary powder blend formulation is shown, comprising
17-HPC as the active pharmaceutical ingredient (API).

| | Content in formulation | |
|---|---|---|
| Components | PGC2011-006B | PGC2011-005B |
| SD powder (PGC2011-020PD, | 1% (100 μg*) | 10% (1000 μg*) |
| Dv(50) = 2.60 μm) | | |
| Lactose Monohydrate: Respitose | 89% | 80% |
| ML001 | | |
| Lactose Monohydrate: Lactohale | 10% | 10% |
| LH300 | | |

*Considering a total fill weight of 10 mg per capsule

TABLE 10
Another exemplary powder blend formulation is shown, comprising
17-HPC as the active pharmaceutical ingredient (API).

| | Content in formulation | |
|---|---|---|
| Components | PFB2011-019B | PFB2011-020B |
| SD powder (PGC2011-022PD, | 1% (100 μg*) | 10% (1000 μg*) |
| Dv(50) = 2.43 μm) | | |
| Lactose Monohydrate: Respitose | 89% | 80% |
| ML001 | | |
| Lactose Monohydrate: Lactohale | 10% | 10% |
| LH300 | | |

*Considering a total fill weight of 10 mg per capsule

### Example 17

Referring to Table 11 (top table, shown below), a summary of different exemplary spray drying (SD) powder blend formulations are shown, comprising 17-HPC as the active pharmaceutical ingredient (API). Table 12 (bottom table, shown below) depicts that an increase in fine-particle dose (FPD) is observed after increasing the fill weight per capsule.

TABLE 11
Summary of different exemplary spray drying (SD) powder blend formulations.

| | | | | | | | Capsules filling | | | | |
|---|---|---|---|---|---|---|---|---|---|---|---|
| SD powder | | | Blend | | | | Shot Weight | | FSI * | | |
| | Dv(50) | | | Theoretical | Assay (% | RSD | Assay | RSD | Fill Weight | FPF (% | FPD (μg |
| Batch | (μm) | Span | Batch | weight (μg API) | API) n = 3 | (%) | (%) n = 3 | (%) | (mg/cap) | total mass) | API) |
| PGC2011- | 3.56 | 2.02 | LBC2011-048B | 100 | 104.53 | 1.12 | 98.0 | 5.1 | 10 (n = 10) | 37.93 | 39.65 |
| 018PD | | | LBC2011-049B | 1000 | 98.49 | 3.91 | 99.8 | 5.1 | 10 (n = 5) | 25.32 | 249.35 |
| PGC2011- | 2.60 | 1.66 | PGC2011-006B | 100 | 101.21 | 1.79 | — | — | 10 (n = 10) | 40.16 | 40.64 |
| 020PD | | | PGC2011-005B | 1000 | 99.99 | 2.51 | — | — | 10 (n = 5) | 33.29 | 332.88 |
| PGC2011- | 2.43 | 1.57 | PFB2011-019B | 100 | 93.05 | 1.14 | — | — | 10.75 (n = 10) | 46.01 | 46.01 |
| 022PD | | | PFB2011-020B | 1000 | 96.03 | 0.59 | — | — | 10 (n = 5) ^{[A]} | 42.67 | 397.00 |

* Use of Plastiape RS-01 Model 7RH device at 4 Kpa
^{[A]} Just for testing purpose 5 capsules were filled with 13.80 mg

TABLE 12
An increase in fine-particle dose (FPD) is observed after increasing
the fill weight per capsule.

| Fill Weight | FPD |
|---|---|
| (mg) | (μg API) |
| 10 (n = 5) | 397.00 |
| 13.80 (n = 5) | 466.00 |

What is claimed is:

1. A pharmaceutical composition for inhalation delivery, comprising: a powder blend comprising a therapeutically effective amount of at least one steroid hormone (progestogen) as a glucocorticoid sensitizer, and at least one pharmaceutically acceptable excipient, wherein the at least one steroid hormone (progestogen) has a particle size distribution profile ranging from about one nanometer to about ten microns in the powder blend.

2. The pharmaceutical composition of claim 1, wherein the at least one steroid hormone comprises 17alpha-hydroxyprogesterone caproate (17-HPC).

3. The pharmaceutical composition of claim 1, wherein the composition has the form of a physical mixture.

4. The pharmaceutical composition of claim 1, wherein the at least one pharmaceutically acceptable excipient comprises lactose.

5. The pharmaceutical composition of claim 1, wherein the composition comprises from about five to about fifty weight percent of the excipient.

6. The pharmaceutical composition of claim 1, wherein the excipient has a particle size distribution profile of from about fifteen microns to about five-hundred microns.

7. The pharmaceutical composition of claim 1, wherein the composition for inhalation delivery is administered by an aerosol spray, a powder mixture in a pressurized pack, a nebulizer or an inhaler.

8. The pharmaceutical composition of claim 2, wherein the composition for inhalation delivery comprises a substantially dry powder comprising 17-HPC after particle size reduction in the range of about 0.001 micron to about 10 microns present in a dry bulking powder suitable for dry powder inhalation, wherein the particle size reduction of hydrophobic 17-HPC can be achieved by milling in water, with a surfactant or without a surfactant, wherein the particle size reduction of 17-HPC is achieved without changing its basic crystalline structure and without generating any additional impurity.

9. The pharmaceutical composition of claim 1, wherein the composition for inhalation delivery comprises a suspension suitable for nebulization.

10. The pharmaceutical composition of claim 1, wherein the composition for inhalation delivery comprises an aerosol propellant suitable for use in a metered dose inhaler.

11. The pharmaceutical composition of claim 2, wherein the composition for inhalation delivery is characterized by a fine-particle dose of 17-HPC in the range of about fifteen to about six-hundred micrograms, wherein smaller particle size correlates with greater fine-particle dose, further wherein the composition is characterized by an approximate blend homogeneity having a relative standard deviation less than about five percent, further wherein the composition for inhalation delivery has a fine particle fraction of about thirty percent or greater.

12. A pharmaceutical composition for inhalation delivery, comprising: a substantially dry powder blend comprising a therapeutically effective amount of at least one steroid hormone (progestogen) as a glucocorticoid sensitizer, and at least one pharmaceutically acceptable excipient, wherein the at least one steroid hormone (progestogen) has a particle size distribution profile ranging from about one nanometer to about ten microns in the powder blend, wherein the composition has the form of a physical mixture and comprises from about five to about fifty weight percent of the excipient, and further wherein the excipient has a particle size distribution of from about fifteen to about five-hundred microns.

13. A method for the treatment of glucocorticoid insensitivity, restoring corticosteroid sensitivity, enhancing glucocorticoid sensitivity or reversing the glucocorticoid insensitivity in a subject experiencing corticosteroid dependence or corticoid resistance or unresponsiveness or intolerance to corticosteroids, comprising administering the pharmaceutical composition of claim 1 comprising a steroid hormone (progestogen) as a glucocorticoid sensitizer to the subject exhibiting one or more glucocorticoid insensitivity related conditions, wherein the subject has no history of menstrual cycle-related exacerbation, and further wherein the glucocorticoid insensitivity related conditions comprise a range of immune-inflammatory disorders/diseases treated with steroids when the therapy fails to achieve disease control or is not effective or intolerant or dependent to corticosteroids, and combinations thereof.

14. A method for the treatment of glucocorticoid insensitivity related diseases, or disorders, or conditions, the method comprising: administering the pharmaceutical composition of claim 1 comprising a steroid hormone (progestogen) as a glucocorticoid sensitizer to a subject exhibiting the glucocorticoid insensitivity, wherein the glucocorticoid insensitivity comprises corticosteroid dependence or corticoid resistance or unresponsiveness or intolerance to corticosteroids, and wherein the subject has no history of menstrual cycle-related exacerbation.

15. The method of claim 14, wherein the effects of the administration of the glucocorticoid sensitizer include, but are not limited to, steroid-sparing in corticosteroid-dependent patients, better responsiveness or tolerance to corticosteroids, achieving efficacy by using a lower dose of corticosteroid, preventing individuals at risk from developing refractory or resistance or exacerbations in response to antigen exposures, infections, exercise, or irritants, achieving optimal immune-functions, easier administrations of steroids that can be tapered or withdrawn, or reducing intolerance to prolonged administration of corticosteroids, decreased risks for developing corticosteroid-related adverse events such as opportunistic infections and bone loss, and combinations thereof.

16. A method for restoring corticosteroid sensitivity or reversing the glucocorticoid insensitivity or enhancing glucocorticoid sensitivity to treat one or more glucocorticoid insensitivity related conditions selected from the group consisting of a range of corticoid resistant diseases and immune-inflammatory disorders treated with glucocorticoid when the therapy becomes ineffective or intolerant or dependent or unresponsive or refractory to corticosteroids, and combinations thereof, the method comprising administering a pharmaceutical composition comprising a steroid hormone to a subject having no history of menstrual cycle-related exacerbation and wherein the subject exhibits one or more glucocorticoid insensitivity related diseases, disorders, or conditions selected from the group consisting of cigarette smoking-related lung diseases such as chronic obstructive pulmonary disease, Asthma, Chronic Bronchitis, Emphysema, Influenza, Acute Non-Influenzal Respiratory Disease, Pneumonia, Tuberculosis, lung cancer, interstitial lung disease, including respiratory bronchiolitis, desquamative interstitial pneumonitis, pulmonary Langerhans cell histiocytosis and combined pulmonary fibrosis and emphysema (CPFE), glucocorticoid resistant asthma, refractory rheumatoid arthritis, refractory inflammatory bowel disease, acute respiratory distress syndrome, interstitial pulmonary fibrosis, cystic fibrosis, refractory ulcerative colitis, children with severe Crohn disease, corticosteroid refractory asthma, desquamative interstitial pneumonia refractory to corticosteroid, refractory inflammatory myopathies, refractory myasthenia gravis, refractory pemphigus vulgaris, methotrexate-refractory RA patients, refractory nephrotic syndrome, refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, critically ill patients with sepsis or acute respiratory distress syndrome (ARDS) and relative adrenal insufficiency, rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, corticosteroid dependent systemic lupus erythematosus, corticosteroid dependent multiple sclerosis, symptomatic corticosteroid dependent asthma, primary Sjogren's syndrome, systemic vasculitis, polymyositis, organ transplants, graft-versus-host disease, inflammatory diseases, autoimmune diseases, hyperproliferative diseases, lupus, osteoarthritis, rhinosinusitis, polyarteritis nodosa, Wegener's granulomatosis, giant cell arteritis, allergic rhinitis, urticaria, hereditary angioedema, tendonitis, bursitis, autoimmune chronic active hepatitis, cirrhosis, transplant rejection, psoriasis, dermatitis, malignancies, leukemia, myelomas, lymphomas, acute adrenal insufficiency, rheumatic fever, granulomatous disease, immune proliferation/apotosis, hypothalamic-pituitary-adrenal (HPA) axis suppression and regulation, hypercortisolemia, modulation of the Th1/Th2 cytokine balance, chronic kidney disease, spinal cord injury, cerebral edema, thrombocytopenia, Little's syndrome, Addison's disease, autoimmune hemolytic anemia, uveitis, pemphigus vulgaris, nasal polyps, sepsis, bacterial infections, viral infections, rickettsial infections, parasitic infections, type II diabetes, obesity, metabolic syndrome, depression, schizophrenia, mood disorders, Cushing's syndrome, anxiety, sleep disorders, memory and learning enhancement, glucocorticoid-induced glaucoma, atopic dermatitis, drug hypersensitivity reactions, serum sickness, bullous dermatitis herpetiformis, contact dermatitis, exfoliative erythroderma, mycosis fungoides, pemphigus, nonsuppurative thyroiditis, sympathetic ophthalmia, uveitis, ocular inflammatory conditions unresponsive to topical steroids, allergic bronchopulmonary aspergillosis, fulminating or disseminated pulmonary tuberculosis when used concurrently with appropriate chemotherapy, hypersensitivity pneumonitis, idiopathic bronchiolitis obliterans with organizing pneumonia, idiopathic eosinophilic pneumonias, idiopathic pulmonary fibrosis, pneumocystis carinii pneumonia (PCP) associated with hypoxemia occurring in an HIV(+) individual who is also under treatment with appropriate anti-PCP antibiotics, a diuresis or remission of proteinuria in nephrotic syndrome, without uremia, of the idiopathic type or that due to lupus erythematosus, ankylosing spondylitis, polymyalgia rheumatic, psoriatic arthritis, relapsing polychondritis, trichinosis with neurologic or myocardial involvement, and tuberculous meningitis.

17. The method of claim 13, further comprising evaluating whether the subject exhibits the one or more glucocorticoid insensitivity related diseases, disorders, or conditions.

18. The method of claim 13, wherein the steroid hormone is a progestogen.

19. The method of claim 13, wherein the pharmaceutical composition is designed to inhibit or delay the onset of the disease or disorder, reduce the risk of relapse, achieve a full or partial reduction of the symptoms or disease state, and alleviate, ameliorate, lessen, or cure the disease, disorder or symptoms.

20. The method of claim 13, wherein the subject is male or female at any age and exhibits the one or more glucocorticoid insensitivity related diseases, disorders, or conditions.

21. The method of claim 13, wherein the pharmaceutical composition is administered daily.

22. The method of claim 13, wherein the pharmaceutical composition is administered according to a dosing regimen selected from the group consisting of an administration interval less than one week, once weekly or exceeding once per week.

23. The method of claim 22, wherein the interval is selected from the group consisting of once per day, twice per day, three times per day, up to 24 times per day, and continuous infusion.

24. The method of claim 22, wherein the interval is selected from the group consisting of once every other week, once monthly, once every two months, and once every three months.

25. The method of claim 13, wherein the pharmaceutical composition is administered once monthly.

26. The method of claim 13, wherein the pharmaceutical composition is administered systemically or locally delivered by a method selected from the group consisting of parenteral, intravenous, pulmonary, oral, rectal, buccal, transdermal, intravaginal delivery, local application, topical application, depot injection, subcutaneous, intraperitoneal, intraarterial and intramuscular injection.

27. The method of claim 26, wherein the transdermal delivery is administered by a formulation selected from the group consisting of a patch, cream, gel, and spray.

28. The method of claim 26, wherein the intravaginal delivery is administered by a formulation selected from the group consisting of a suppository, gel, and cream.

29. The method of claim 26, wherein the pulmonary drug delivery is administered by a formulation selected from the group consisting of a medical aerosol and an inhalant delivery device.

30. The method of claim 13, wherein the progestogen is a progestin.

31. The method of claim 13, wherein the progestogen is selected from the group consisting of progesterone, retroprogesterone, progesterone derivative, 17alpha-OH progesterone derivatives (both pregnanes and norpregnanes), 19-norprogesterone derivatives, 19-nortestosterone derivatives (both estranges and gonanes), spironolactone derivatives, and a combination thereof.

32. The method of claim 13, wherein the progestogen is selected from the group consisting of 17alpha-hydroxyprogesterone or a derivative thereof, natural progesterone, dydrogesterone or a derivative or metabolite thereof, medrogestone or a derivative or metabolite thereof, medroxyprogesterone or a derivative or metabolite thereof, megestrol or a derivative or metabolite thereof, chlormadinone or a derivative or metabolite thereof, cyproterone or a derivative or metabolite thereof, gestonorone or a derivative or metabolite thereof, nomegestrol or a derivative or metabolite thereof, demegestone or a derivative or metabolite thereof, promegestone or a derivative or metabolite thereof, nestorone or a derivative or metabolite thereof, trimegestone or a derivative or metabolite thereof, norethisterone, norethisterone or a derivative or metabolite thereof, lynestrenol or a derivative or metabolite thereof, ethynodiol or a derivative or metabolite thereof, diacetate, norgestrel or a derivative or metabolite thereof, levonorgestrel or a derivative or metabolite thereof, desogestrel or a derivative or metabolite thereof, etonogestrel (3-keto-desogestrel) or a derivative or metabolite thereof, gestodene or a derivative or metabolite thereof, norgestimate or a derivative or metabolite thereof, norelgestromin (17-deacetyl norgestimate) or a derivative or metabolite thereof, dienogest or a derivative or metabolite thereof, drospirenone or a derivative or metabolite thereof, norethindrone or a derivative or metabolite thereof, norethynodrel or a derivative or metabolite thereof, norgestrel or a derivative or metabolite thereof, drospirenone or a derivative thereof, or metabolite or a derivative thereof, etonogestrel or a derivative or metabolite thereof, 19-nortestosterone or a derivative or metabolites thereof, dienogest or a derivative or metabolite thereof, norethynodrel or a derivative or metabolite thereof, cyproterone or a derivative or metabolite thereof, tibolone or a derivative or metabolite thereof, 19-norprogesterone or a derivative or metabolite thereof, and combinations thereof.

33. The method of claim 32, wherein the derivative of progestogen is selected from the group consisting of an amine salt, alkali metal salt, transition metal salt, other metal salt, salt of a mineral acid, salt of an organic acid, an ester, enol ether or ester, acid, base, solvate, hydrate or prodrug prior to formulation.

34. The method of claim 33, wherein the derivative of 17alpha-hydroxyprogesterone is a carboxylic acid ester of 17alpha-hydroxyprogesterone.

35. The method of claim 33, wherein the derivative of medroxyprogesterone is medroxyprogesterone acetate, the derivative of megestrol is megestrol acetate, the derivative of chlormadinone is chlormadinone acetate, the derivative of cyproterone is cyproterone acetate, the derivative of gestonorone is gestonorone caproate, the derivative of nomegestrol is nomegestrol acetate, the derivative of norethisterone is norethisterone acetate, and the derivative of ethynodiol is ethynodiol diacetate.

36. The method of claim 13, wherein the progestogen is administered prior to, simultaneously with, or following the administration of glucocorticoid.

37. The method of claim 13, wherein the glucocorticoid is selected from the group consisting of hydrocortisone (cortisol), cortisone acetate, dexamethasone, prednisone, prednisolone, methylprednisolone, betamethasone, triamcinolone, beclometasone, Paramethasone, fluticasone, fludrocortisone acetate, deoxycorticosterone acetate (DOCA), Fluprednisolone, fluticasone propionate, budesonide, beclomethasone dipropionate, flunisolide and triamcinolone acetonide.

38. The method of claim 13, further comprising one or more additional treatments for restoring corticosteroid sensitivity or reversing the glucocorticoid insensitivity or enhancing glucocorticoid sensitivity to treat one or more glucocorticoid insensitivity related conditions.

39. The method of claim 38, wherein the one or more additional treatments are selected from the group consisting of an androgen, an estrogen, immunosuppressive or immunomodulator agent, calcineurin inhibitor, p38 MAP kinase inhibitor, JNK inhibitor, Vitamin D, MIF inhibitor, Histone deacetylate-2 activator, Theophylline, Phosphoinositide-3-kinase-δ inhibitor, antioxidant, iNOS inhibitor, muscarinic receptor antagonist, bronchodilators, long-acting beta-agonists, anti-leukotrienes, anticholinergic agents, narrow spectrum kinase inhibitors, P-glycoprotein inhibitor, and combinations thereof.

40. The method of claim 38, wherein the progestogen is administered prior to, simultaneously with, or following the administration of an agent selected from the group consisting of dehydroepiandrosterone (DHEA), estradiol, cyclosporine, methotrexate, gold, 6-mercaptopurine, infliximab, etanercept, adalimumab, intravenous immunoglobulin, Mepolizumab, cyclosporin, tacrolimus, p38 MAP kinase inhibitor, JNK inhibitor, Vitamin D, MIF inhibitor, Histone deacetylate-2 activator, Theophylline, Phosphoinositide-3-kinase-δ inhibitor, antioxidant, iNOS inhibitor, muscarinic receptor antagonist, bronchodilators, long-acting beta-agonists, anti-leukotrienes, anticholinergic agents, narrow spectrum kinase inhibitors, P-glycoprotein inhibitor, and combinations thereof.

41. The method of claim 13, wherein the pharmaceutical composition is administered to the subject via oral ingestion, further wherein the composition comprises from about 0.001 to 100 mg/kg of body weight of progestogen given orally per day.

42. A kit comprising a pharmaceutical composition, wherein the pharmaceutical composition comprises a steroid hormone, one or more pharmaceutically acceptable excipients, and instructions for administering the pharmaceutical composition to a subject having no history of menstrual cycle-related exacerbation, wherein the subject exhibits one or more glucocorticoid insensitivity related conditions.

43. A kit comprising a pharmaceutical composition, wherein the pharmaceutical composition comprises a steroid hormone, one or more pharmaceutically acceptable excipients, and instructions for administering the pharmaceutical composition as a glucocorticoid sensitizer to a subject exhibiting glucocorticoid insensitivity, wherein the subject has no history of menstrual cycle-related exacerbation.

44. A kit comprising a pharmaceutical composition, wherein the pharmaceutical composition comprises a steroid hormone, one or more pharmaceutically acceptable excipients, and instructions for administering the pharmaceutical composition as a glucocorticoid sensitizer to achieve the effects of steroid-sparing in corticosteroid-dependent patients, better responsiveness or tolerance to corticosteroids, achieving efficacy by using a lower dose of corticosteroid, preventing individuals at risk for developing refractory or resistance or exacerbations in response to antigen exposures, infections, exercise, or irritants, achieving optimal immune-functions, easier steroid tapered or withdrawn, or prolonged administration of corticosteroids, decreased risks for developing corticosteroid-related adverse events such as opportunistic infections and bone loss, and combinations thereof.

45. A kit comprising a pharmaceutical composition, wherein the pharmaceutical composition comprises a steroid hormone and one or more pharmaceutically acceptable excipients; and instructions for administering the pharmaceutical composition to a subject to treat one or more glucocorticoid insensitivity related conditions selected from the group consisting of corticoid resistant diseases and immune-inflammatory disorders treated with glucocorticoid when the therapy becomes ineffective or intolerant or dependent or unresponsive or refractory to corticosteroids, and combinations thereof, wherein the subject has no history of menstrual cycle-related exacerbation and exhibits one or more glucocorticoid insensitivity related diseases, disorders, or conditions selected from the group consisting of cigarette smoking-related lung diseases such as chronic obstructive pulmonary disease, Asthma, Chronic Bronchitis, Emphysema, Influenza, Acute Non-Influenzal Respiratory Disease, Pneumonia, Tuberculosis, lung cancer, interstitial lung disease, including respiratory bronchiolitis, desquamative interstitial pneumonitis, pulmonary Langerhans cell histiocytosis and combined pulmonary fibrosis and emphysema (CPFE), glucocorticoid resistant asthma, refractory rheumatoid arthritis, refractory inflammatory bowel disease, acute respiratory distress syndrome, interstitial pulmonary fibrosis, cystic fibrosis, refractory ulcerative colitis, severe Crohn's disease, corticosteroid refractory asthma, desquamative interstitial pneumonia refractory to corticosteroid, refractory inflammatory myopathies, refractory myasthenia gravis, refractory pemphigus vulgaris, methotrexate-refractory RA patients, refractory nephrotic syndrome, refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, sepsis, acute respiratory distress syndrome (ARDS), relative adrenal insufficiency, rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, corticosteroid dependent systemic lupus erythematosus, corticosteroid dependent multiple sclerosis, symptomatic corticosteroid dependent asthma, primary Sjogren's syndrome, systemic vasculitis, polymyositis, organ transplants, graft-versus-host disease, inflammatory diseases, autoimmune diseases, hyperproliferative diseases, lupus, osteoarthritis, rhinosinusitis, polyarteritis nodosa, Wegener's granulomatosis, giant cell arteritis, allergic rhinitis, urticaria, hereditary angioedema, tendonitis, bursitis, autoimmune chronic active hepatitis, cirrhosis, transplant rejection, psoriasis, dermatitis, malignancies, leukemia, myelomas, lymphomas, acute adrenal insufficiency, rheumatic fever, granulomatous disease, immune proliferation/apotosis, hypothalamic-pituitary-adrenal (HPA) axis suppression and regulation, hypercortisolemia, modulation of the Th1/Th2 cytokine balance, chronic kidney disease, spinal cord injury, cerebral edema, thrombocytopenia, Little's syndrome, Addison's disease, autoimmune hemolytic anemia, uveitis, pemphigus vulgaris, nasal polyps, sepsis, bacterial infections, viral infections, rickettsial infections, parasitic infections, type II diabetes, obesity, metabolic syndrome, depression, schizophrenia, mood disorders, Cushing's syndrome, anxiety, sleep disorders, memory and learning enhancement, glucocorticoid-induced glaucoma, atopic dermatitis, drug hypersensitivity reactions, serum sickness, bullous dermatitis herpetiformis, contact dermatitis, exfoliative erythroderma, mycosis fungoides, pemphigus, bullous pemphigoid, nonsuppurative thyroiditis, sympathetic ophthalmia, uveitis, ocular inflammatory conditions unresponsive to topical steroids, allergic bronchopulmonary aspergillosis, fulminating or disseminated pulmonary tuberculosis when used concurrently with appropriate chemotherapy, hypersensitivity pneumonitis, idiopathic bronchiolitis obliterans with organizing pneumonia, idiopathic eosinophilic pneumonias, idiopathic pulmonary fibrosis, pneumocystis carinii pneumonia (PCP) associated with hypoxemia occurring in an HIV(+) individual who is also under treatment with appropriate anti-PCP antibiotics, a diuresis or remission of proteinuria in nephritic syndrome, without uremia, of the idiopathic type or that due to lupus erythematosus, ankylosing spondylitis, polymyalgia rheumatic, psoriatic arthritis, relapsing polychondritis, trichinosis with neurologic or myocardial involvement, and tuberculous meningitis.

46. The kit of claim 42, further comprising instructions for evaluating whether the subject exhibits the one or more glucocorticoid insensitivity related diseases, disorders, or conditions.

47. A kit comprising a pharmaceutical composition comprising a steroid hormone and one or more pharmaceutically acceptable excipients; and instructions for administering the pharmaceutical composition.

48. The kit of claim 42, wherein the steroid hormone is a progestogen.

49. The kit of claim 42, wherein the pharmaceutical composition is designed to inhibit or delay the onset of the disease or disorder, reduce the risk of relapse, achieve a full or partial reduction of the symptoms or disease state, or alleviate, ameliorate, lessen, or cure the disease or disorder and/or its symptoms.

50. The kit of claim 42, wherein the subject is male or female of any age, and exhibits the one or more glucocorticoid insensitivity related diseases, disorders, or conditions.

51. The kit of claim 42, wherein the instructions comprise instructions for administering the pharmaceutical composition daily.

52. The kit of claim 42, wherein the instructions comprise instructions for administering the pharmaceutical composition at an interval less than one week, once weekly or exceeding once per week.

53. The kit of claim 42, wherein the instructions comprise instructions for administering the pharmaceutical composition at an interval selected from the group consisting of once per day, twice per day, three times per day, up to 24 times per day, and by continuous infusion.

54. The kit of claim 42, wherein the instructions comprise instructions for administering the pharmaceutical composition at an interval selected from the group consisting of once every other week, once monthly, once every two months, and once every three months.

55. The kit of claim 42, wherein the instructions comprise instructions for administering the pharmaceutical composition once monthly.

56. The kit of claim 42, wherein the instructions comprise instructions for administering the pharmaceutical composition by a method selected from the group consisting of parenteral, intravenous, pulmonary, oral, rectal, buccal, transdermal, intravaginal delivery, local application, topical application, depot injection, subcutaneous, intraperitoneal, intraarterial, and intramuscular injection.

57. The kit of claim 56, wherein the instructions comprise instructions for administering the pharmaceutical composition transdermally via a formulation selected from the group consisting of a patch, cream, gel, and spray.

58. The kit of claim 56, wherein the instructions comprise instructions for administering the pharmaceutical composition intravaginally via a formulation selected from the group consisting of a suppository, gel, and cream.

59. The kit of claim 56, wherein the instructions comprise instructions for administering the pharmaceutical composition pulmonarily via a formulation selected from the group consisting of an inhaled medical aerosol and an inhalant delivery device.

60. The kit of claim 42, wherein the progestogen is a progestin.

61. The kit of claim 42, wherein the progestogen is selected from the group consisting of progesterone, retroprogesterone, progesterone derivative, 17α-OH progesterone derivatives (both pregnanes and norpregnanes), 19-norprogesterone derivatives, 19-nortestosterone derivatives (both estranges and gonanes), spironolactone derivatives, and a combination thereof.

62. The kit of claim 42, wherein the progestogen is selected from the group consisting of 17alpha-hydroxyprogesterone or a derivative thereof, natural progesterone, dydrogesterone or a derivative or metabolite thereof, medrogestone or a derivative or metabolite thereof, medroxyprogesterone or a derivative or metabolite thereof, megestrol or a derivative or metabolite thereof, chlormadinone or a derivative or metabolite thereof, cyproterone or a derivative or metabolite thereof, gestonorone or a derivative or metabolite thereof, nomegestrol or a derivative or metabolite thereof, demegestone or a derivative or metabolite thereof, promegestone or a derivative or metabolite thereof; nestorone or a derivative or metabolite thereof, trimegestone or a derivative or metabolite thereof, norethisterone or a derivative or metabolite thereof, lynestrenol or a derivative or metabolite thereof, ethynodiol or a derivative or metabolite thereof, norgestrel or a derivative or metabolite thereof, levonorgestrel or a derivative or metabolite thereof, desogestrel or a derivative or metabolite thereof, etonogestrel (3-keto-desogestrel) or a derivative or metabolite thereof, gestodene or a derivative or metabolite thereof, norgestimate or a derivative or metabolite thereof, norelgestromin (17-deacetyl norgestimate) or a derivative or metabolite thereof, dienogest or a derivative or metabolite thereof, drospirenone or a derivative or metabolite thereof, norethindrone or a derivative or metabolite thereof, norethynodrel or a derivative or metabolite thereof, norgestrel or a derivative or metabolite thereof, drospirenone or a derivative or metabolite thereof, etonogestrel or a derivative or metabolite thereof, 19-nortestosterone or a derivative or metabolite thereof, dienogest or a derivative or metabolite thereof, norethynodrel or a derivative or metabolite thereof, cyproterone or a derivative or metabolite thereof, tibolone or a derivative or metabolite thereof, 19-norprogesterone or a derivative or metabolite thereof, and combinations thereof.

63. The kit of claim 42, wherein the progestogen is selected from the group consisting of the corresponding progestogen amine salt, alkali metal salt, transition metal salt, other metal salt, salt of a mineral acid, salt of an organic acid, ester, enol ether or ester, acid, base, solvate, hydrate, and prodrug prior to formulation.

64. The kit of claim 63, wherein the derivative of 17alpha-hydroxyprogesterone is a carboxylic acid ester of 17alpha-hydroxyprogesterone.

65. The kit of claim 63, wherein the derivative of medroxyprogesterone is medroxyprogesterone acetate, the derivative of megestrol is megestrol acetate, the derivative of chlormadinone is chlormadinone acetate; the derivative of cyproterone is cyproterone acetate, the derivative of gestonorone is gestonorone caproate, the derivative of nomegestrol is nomegestrol acetate, the derivative of norethisterone is norethisterone acetate, and the derivative of ethynodiol is ethynodiol diacetate.

66. The kit of claim 42, wherein the instructions comprise instructions for administering the progestogen prior to, simultaneously with, or following the administration of glucocorticoid.

67. The kit of claim 66, wherein the derivative of glucocorticoid is selected from the group consisting of hydrocortisone (cortisol), cortisone acetate, dexamethasone, prednisone, prednisolone, methylprednisolone, betamethasone, triamcinolone, beclometasone, paramethasone, fluticasone, fludrocortisone acetate, deoxycorticosterone acetate (DOCA), Fluprednisolone, fluticasone propionate, budesonide, beclomethasone dipropionate, flunisolide and triamcinolone acetonide.

68. The kit of claim 42, wherein the instructions comprise instructions for providing one or more additional treatments for restoring corticosteroid sensitivity or reversing the glucocorticoid insensitivity or enhancing glucocorticoid sensitivity to treat one or more glucocorticoid insensitivity related conditions.

69. The kit of claim 68, wherein the one or more additional treatments are selected from the group consisting of an androgen, an estrogen, immunosuppressive or immunomodulator agent, calcineurin inhibitor, p38 MAP kinase inhibitor, JNK inhibitor, Vitamin D, MIF inhibitor, histone deacetylate-2 activator, theophylline, phosphoinositide-3-kinase-δ inhibitor, antioxidant, iNOS inhibitor, muscarinic receptor antagonist, bronchodilators, long-acting beta-agonists, anti-leukotrienes, anticholinergic agents, narrow spectrum kinase inhibitors, p-glycoprotein inhibitor, and combinations thereof.

70. The kit of claim 42, wherein the instructions comprise instructions for administering the progestogen prior to, simultaneously with, or following the administration of an agent selected from the group consisting of dehydroepiandrosterone (DHEA), estradiol, cyclosporine, methotrexate, gold, 6-mercaptopurine, infliximab, etanercept, adalimumab, intravenous immunoglobulin, Mepolizumab, cyclosporin, tacrolimus, p38 MAP kinase inhibitor, JNK inhibitor, Vitamin D, MIF inhibitor, Histone deacetylate-2 activator, Theophylline, Phosphoinositide-3-kinase-δ inhibitor, antioxidant, iNOS inhibitor, P-glycoprotein inhibitor, and combinations thereof.

71. The kit of claim 42, comprising a pharmaceutical composition comprising a steroid hormone and one or more pharmaceutically acceptable excipients; and instructions for administering the pharmaceutical composition to the subject via oral ingestion, wherein the composition comprises from about 0.001 to 100 mg/kg of body weight of progestogen given orally per day, and further wherein the amount used with non-oral routes is determined based upon corresponding serum concentration level of an oral dosage or containing a quantity of the active compound in an amount sufficient to alleviate the symptoms of the treated subject.

72. A method of treating a smoking-induced glucocorticoid resistance disease, comprising administering at least one progestogen.

73. The method of claim 72, wherein the progestogen is selected from the group consisting of 17HPC, P4 and MPA.

74. The method of claim 72, wherein the disease is chronic obstructive pulmonary disease (COPD), and other smoking-related lung diseases such as chronic obstructive pulmonary disease, Asthma, Chronic Bronchitis, Emphysema, Influenza, Acute Non-Influenzal Respiratory Disease, Pneumonia, Tuberculosis, lung cancer, interstitial lung disease, including respiratory bronchiolitis, desquamative interstitial pneumonitis, pulmonary Langerhans cell histiocytosis and combined pulmonary fibrosis and emphysema (CPFE).

75. A method for preparing the composition of claim 1, comprising: preparing a powder blend comprising the therapeutically effective amount of at least one steroid hormone (progestogen) as a glucocorticoid sensitizer and the at least one pharmaceutically acceptable excipient, wherein preparation of the powder blend comprises a particle size reduction process,
further wherein the at least one steroid hormone (progestogen) obtained from said particle size reduction process has a particle size distribution profile ranging from about one nanometer to about ten microns in the powder blend.

76. The method of claim 75, wherein the at least one steroid hormone comprises 17-HPC.

77. The method of claim 75, wherein the composition has the form of a physical mixture.

78. The method of claim 75, wherein the at least one pharmaceutically acceptable excipient comprises lactose.

79. The method of claim 75, wherein at least one pharmaceutically acceptable surfactant is employed in the particle size reduction process.

80. The method of claim 75, wherein the at least one pharmaceutically acceptable surfactant comprises Tween 80.

81. The method of claim 75, wherein the Tween 80 is present at a concentration of from about five to about fifteen percent.

82. The method of claim 75, wherein the particle size reduction process is carried out with water and without a surfactant.

83. The method of claim 75, wherein the composition comprises from about five to about fifty weight percent of the excipient.

84. The method of claim 79, wherein the excipient has a particle size distribution of from about fifteen microns to about five-hundred microns.

85. The method of claim 75, wherein the inhalation delivery is administered by an aerosol spray, a powder mixture in a pressurized pack, a nebulizer or an inhaler.

86. The method of claim 75, wherein the composition for inhalation delivery is provided as a substantially dry powder comprising 17-HPC present in a dry bulking powder suitable for dry powder inhalation.

87. The method of claim 75, wherein the composition for inhalation delivery comprises a suspension suitable for nebulization.

88. The method of claim 75, wherein the composition for inhalation delivery comprises an aerosol propellant suitable for use in a metered dose inhaler.

89. The method of claim 75, wherein the particle size distribution profile is obtained from a suspension prepared with the surfactant Tween 80 or with water and without a surfactant.

90. The method of claim 75, wherein the particle size distribution profile is obtained from a 17-HPC dry powder after a spray-drying process.
